(19) United States
(12) Patent Application Publication
(10) Pub. No.: US 2003/0215793 A1
(43) Pub. Date: Nov. 20, 2003

(54) Complete genome sequence of a simian immunodeficiency virus from a wild chimpanzee

(76) Inventors: Beatrice H. Hahn, Birmingham, AL (US); George M. Shaw, Birmingham, AL (US); Mario L. Santiago, Homewood, AL (US); Cynthia M. Rodenburg, Birmingham, AL (US); Shadrack Kamenya, Kigoma (TZ); Frederic Bibollet-Ruche, Birmingham, AL (US); Martin N. Muller, Ann Arbor, MI (US); Anthony Collins, Kigoma (TZ); Richard W. Wrangham, Weston, MA (US); Jane Goodall, Dar Es Salaam (TZ); Paul M. Sharp, Nottingham (GB)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/346,000

(22) Filed: Jan. 16, 2003

Related U.S. Application Data
(60) Provisional application No. 60/349,617, filed on Jan. 17, 2002.

(51) Int. Cl. C12Q 1/70; C07H 21/04; C12N 7/00; C07K 14/14; C12N 5/06; C12N 15/867

(52) U.S. Cl. 435/5; 435/69.3; 435/235.1; 435/363; 536/23.72; 530/350; 435/320.1; 435/456

(57) ABSTRACT

The present disclosure relates to the determination of the complete genomic nucleic acid sequence of a new simian immunodeficiency virus (SIVcpzTAN1) isolated from a wild chimpanzee (Ch-06) from the Gombe National Park in Tanzania and to the nucleic acids derived therefrom. The disclosure also relates to the peptides encoded by and/or derived from the SIVcpzTAN1 nucleic acid sequence, to host cells containing the nucleic acids sequences and/or peptides, to diagnostic kits, immunogens and methods which employ the nucleic acids, peptides and/or host cells of the present disclosure, and to non-invasive methods for the detection of SIVcpz and related viruses from animal species in the wild.

This application claims priority to and benefit of provisional application No. 60/349,617, filed Jan. 17, 2002.

### FIELD OF THE DISCLOSURE

The present disclosure relates to the determination of the complete genomic nucleic acid sequence of a new simian immunodeficiency virus (SIVcpzTAN1) isolated from a wild chimpanzee (Ch-06) and to the nucleic acids derived therefrom. The disclosure also relates to the peptides encoded by and/or derived from the SIVcpzTAN1 nucleic acid sequence, to host cells containing the nucleic acids sequences and/or peptides, to diagnostic kits, immunogens and methods which employ the nucleic acids, peptides and/or host cells of the present disclosure, and to non-invasive methods for the detection of SIVcpz and related viruses from animal species in the wild. SIVcpz TAN1 nucleic acid sequences and peptides encoded by or derived from those sequences can be used for a variety of diagnostic and therapeutic purposes, or may be used to generate vaccines against SIVcpz or HIV-1 or any primate lentivirus related to SIVcpz or HIV-1.

### BACKGROUND

Substantial progress has been made in our understanding of the acquired immunodeficiency syndrome or AIDS. The principal causative agent has been demonstrated to be a non-transforming retrovirus with a tropism for CD4 helper/inducer lymphocytes (84, 85) and it has been estimated that millions of people world-wide have already been infected. Infection with this virus leads, at least in a significant percentage of cases, to a progressive depletion of the CD4 lymphocyte population with a concomitant increasing susceptibility to the opportunistic infections which are characteristic of the disease. Epidemiological studies indicate that human immunodeficiency virus, type 1 (HIV-1), the etiological agent responsible for the majority of AIDS cases, is currently the most widely disseminated HIV worldwide. A second group of human immunodeficiency-associated retroviruses, human immunodeficiency virus type 2 (HIV-2), was identified in West Africa (7, 86).

The simian immunodeficiency viruses (SIVs) are non-human primate lentiviruses that are the closest known relatives of the HIVs. One common characteristic among all naturally occurring SIVs is that none are associated with immunodeficiency or any other disease in their natural hosts (9, 13, 22, 28, 30, 35, and 38). This finding is in marked contrast to AIDS, which occurs in humans and macaques infected with primate lentiviruses (2, 7, 8, 27, 35). This lack of disease in the natural SIV hosts may be an example of long-term evolution toward avirulence (16), which supports the hypothesis that SIV has infected African simians for a relatively long time.

Phylogenetic analyses of SIV isolates reveal that they belong to six distinct lineages of the lentivirus family of retroviruses (47). These six SIV lentiviral lineages form a distinct sub-group because primate viruses are more closely related to each other than to lentiviruses from non-primate hosts (47). Importantly, only simian species indigenous to the African continent are naturally infected (4, 13, 28, 35). Thus far, natural SIV infections in Africa have been documented in 30 some African primates, including the sooty mangabey (SM) (Cercocebus torquatus atys) (SIVsm strains), in Liberia (30), in Sierra Leone (4, 5), and the Ivory Coast (43); in all four sub-species of African green monkeys (agm) (Cercopithecus aethiops) (1, 21, 22, 25, 33, 34, 39) (SIVagm strains), in eastern, central and western Africa; in the Sykes monkey (syk) (Cercopithecus mitis) (SIVsyk strains) in Kenya (9); in the mandrill (mnd) (Mandrillus sphinx) (SIVmnd1 strains) (38, 50) in Gabon; in chimpanzees (cpz) (Pan troglodytes) (SIVcpz strains) (19, 20, 41, 42) from Gabon, Cameroon and the Democratic Republic of Congo, and in colobus (col) monkeys from Cameroon (90). Because these SIVs and their simian hosts are highly divergent from each other and widely distributed across Africa, it is believed that the SIV family evolved and established itself in African simians long before acquired immunodeficiency syndrome (AIDS) appeared in humans (4, 15, 18, 19, 21, 30, 37, 47). Interestingly, the phylogeny of HIV is markedly different from SIV, because genetic analyses have shown that the human viruses do not represent separate seventh or eighth lineages of primate lentiviruses, but instead, are members of two of the six existing SIV lineages (37, 46). HIV-1 falls within the SIVcpz group (19, 51) and HIV-2 falls within the SIVsm family (18, 23). These phylogenetic data have long suggested separate simian origins for HIV-1 and HIV-2 (37, 46).

Serological cross-reactivity has been observed between structural proteins of different HIV/SIVs. At the level of the envelope proteins, cross-reactions exist between envelope proteins of SIVmac, SIVsm, SIVagm and HIV-2, but sera from non-human primates infected with these viruses generally do not react to HIV-1 envelope proteins.

Molecular studies of naturally occurring SIVsm and HIV-2 strains from rural West Africa have provided convincing evidence for a simian origin of HIV-2. A close genetic relationship has been established between the HIV-2 D and E groups and SIVsm strains found in household pet sooty mangabeys in West Africa (4, 14, 15). Moreover, all six known subtypes of HIV-2, including a new subtype F (3), are found only within the natural range of SIVsm-infected sooty mangabeys in West Africa. No other area of Africa or of the world has all six known HIV-2 subtypes. Together, these data provide strong support for independent transmissions of SIVsm from naturally infected sooty mangabeys to humans.

In contrast, there is much less information to support a chimpanzee origin for HIV-1. SIVcpz from west central African chimpanzees (Pan troglodytes troglodytes) is the closest relative to all three major groups of HIV-1 (M, N and O). Because of the relatedness of SIVcpzPtt and HIV-1, chimpanzees from this subspecies (P. t. troglodytes) have been implicated as a reservoir for the human infections. Six different SIVcpz strains have thus far been identified (20, 41, 42, 51). The first one (GAB1) was isolated from a household pet chimpanzee in Gabon (42). Three further SIVcpz strains were isolated from captive chimpanzees in Cameroon (CAM3, CAM4, CAM5), but one of them represents a cage transmission (91). An additional SIVcpz strain (ANT) was found in a captive chimpanzee which was wild caught in the Democratic Republic of Congo and thus likely infected in Africa (41, 51). One more (US) was identified in a wild-caught chimpanzee housed at an American primate center (92). Finally, PCR data suggested the existence of a sixth SIVcpz strain (GAB2), again from a chimpanzee from Gabon (20). All known HIV-1 strains are most closely related to SIVcpzPtt strains. Thus, the hypothesis that HIV-1 is derived from west central African chimpanzees is quite plausible. However, additional SIVs within the HIV-1/SIVcpz lineage must be found to fully understand the origin and evolution of the HIV-1 family. Because all SIVcpz strains identified to date are derived from captive chimpanzees, nothing is known about the prevalence, geographic distribution and genetic diversity of SIVcpz in the wild.

The present disclosure is based on the genetic characterization of a new SIV strain from a wild east African chimpanzee of the subspecies Pan troglodytes schweinfurthii.(83). This disclosure is the first prevalence study and detection of SIVcpz in wild-living apes. The virus has been designated SIVcpzTAN1.

The SIVcpzTAN1 nucleic acid and polypeptide sequence(s) described herein will permit the development of new serological screening assays for testing and detection of a wider range of SIVcpz like viruses in humans and primates. Strain specific reagents (antigens, polypeptides, etc.) are required to test for SIVcpz specific antibodies as a sign of viral infection. Such strain specific antigens can now be designed on the basis of the SIVcpzTAN1 sequence(s) described herein. If evidence is found that humans in Africa are infected with a wider variety of SIVcpz (regardless whether this infection is pathogenic or not), then new screening assays for the world's blood supply will have to be developed. In Gag, Pol and Env proteins,SIVcpzTAN1 differs from SIVcpzPtt strains by 36, 30 and 51% of amino acid sequences (new paper). This degree of genetic diversity may necessitate the development of SIVcpz lineage specific assays. The sequences of TAN1 are necessary to design such strain-specific tests.

Additionally, the SIVcpzTAN1 nucleic acid and polypeptide sequence(s) described herein will permit the development of new vaccine approaches against HIV-1. It is contemplated that evolutionarily conserved peptide sequences between SIVcpzTAN1 and HIV-1 or other primate lentiviruses could be useful in the design and development of protective vaccines against HIV-1, or any primate lentivirus related to SIVcpz or HIV-1.

### SUMMARY OF THE DISCLOSURE

The present disclosure pertains to the isolation and characterization of the genomic sequence of SIVcpzTAN1, a new simian immunodeficiency virus identified from a wild east African chimpanzee Pan troglodytes schweinfurthii, (designated Ch-06) identified in Gombe National Park, Tanzania and nucleic acids derived therefrom.

In particular, the present disclosure relates to nucleic acids comprising the complete genomic sequence of SIVcpzTAN1, as well as nucleic acids comprising the complementary (or antisense) sequence of the genomic sequence of SIVcpzTAN1, and nucleic acids derived therefrom.

The disclosure also relates to vectors comprising the nucleic acid genomic sequence of SIVcpzTAN1, as well as vectors comprising nucleic acids comprising the complementary (or antisense) sequence of the genomic sequence of SIVcpzTAN1, and nucleic acids derived therefrom.

The disclosure also relates to cultured host cells comprising the nucleic acid genomic sequence of SIVcpzTAN1, as well as host cells comprising nucleic acids comprising the complementary (or antisense) sequence of the genomic sequence of SIVcpzTAN1, and nucleic acids derived therefrom.

The disclosure also relates to host cells containing vectors comprising the genomic sequence of SIVcpzTAN1, as well as host cells containing vectors comprising nucleic acids comprising the complementary (or antisense) sequence of the genomic sequence of SIVcpzTAN1, and nucleic acids derived therefrom.

The disclosure also relates to synthetic or recombinant polypeptides encoded by or derived from the nucleic acid sequence of the genome of SIVcpzTAN1, and fragments thereof.

The disclosure also relates to methods for producing the polypeptides of the disclosure in culture using the SIVcpzTAN1 virus or nucleic acids derived therefrom, including recombinant methods for producing the polypeptides of the invention.

The disclosure further relates to methods of using the polypeptides of the disclosure as immunogens to stimulate an immune response in humans or other mammals, such as the production of antibodies, or the generation of cytotoxic or helper T-lymphocytes.

The disclosure also relates to methods for the use of the nucleic acids and polypeptides of the disclosure to develop vaccines against HIV-1, or any primate lentivirus related to SIVcpz or HIV-1.

The disclosure also relates to methods of using the polypeptides of the disclosure to detect antibodies which immunologically react with the SIVcpzTAN1 virion and/or its encoded polypeptides, in a mammal or in a biological sample.

The disclosure also relates to kits for the detection of antibodies specific for SIVcpzTAN1 in a biological sample where said kit contains at least one polypeptide encoded by or derived from the SIVcpzTAN1 nucleic acid sequences of the disclosure.

The disclosure also relates to antibodies which immunologically react with the SIVcpzTAN1 virion and/or its encoded polypeptides.

The disclosure also relates to methods of detecting SIVcpzTAN1 virion and/or its encoded polypeptides, or fragments thereof, using the antibodies of the disclosure. The disclosure also relates to kits for detecting SIVcpzTAN1 virion, and/or its encoded polypeptides, wherein the kit comprises at least one antibody of the invention.

The disclosure also relates to a method for detecting the presence of SIVcpzTAN1 virus in a mammal or a biological sample, said method comprising analyzing the DNA or RNA of a mammal or a sample for the presence of the RNAs, cDNAs or genomic DNAs which will hybridize to a nucleic acid derived from SIVcpzTAN1.

### BRIEF DESCRIPTION OF THE FIGURES

FIG. 1A shows a Western blot of urine samples taken from wild-living chimpanzees and captive chimpanzees of known SIVcpz status. The Western blot was performed as described in Example 1. The Western blot illustrates urine samples taken from two captive chimpanzees infected with SIVcpz designated as CAM4 and ch-No, a wild-living chimpanzee (Ch-06) determined to be infected with SIVcpzTAN1, and from several wild-living chimpanzees determined not to be infected with SIVcpz designated Ch-01 through Ch-05.

FIG. 1B shows RNA extracted from fecal samples and analyzed by diagnostic PCR as described in Example 1. PCR products were separated by Gel electrophoresis and visualized. FIG. 1B shows a marker (designated M), a positive control and a negative control (designated + and −, respectively) and samples from a wild-living chimpanzee (Ch-06) determined to be infected with SIVcpzTAN1, and from several wild-living chimpanzees determined not to be infected with SIVcpz designated Ch-01, Ch-03 and Ch-05.

FIG. 2 shows phylogenetic trees of SIVcpzTAN1 Gag, Pol and Env amino acid sequences and other SIVcpz and HIV-1 strains. The asterisks denote >95% bootstrap values.

FIG. 3 shows the alignment of the Vpu amino acid sequences derived from HIVcpzTAN1 and HIVcpzANT, illustrating a significant amount of diversity even between two closely related HIVcpz strains. Identical amino acids are indicated by asterisks. It should be noted that despite the high degree of divergence between these two sequences, TAN1 did show conservation of two serine residues critical for Vpu-induced CD4 degradation (indicated by arrows).

FIG. 4 shows lineage specific protein signatures of HIVcpzTAN1 and SIVcpzANT. Allignments of the indicated SIVcpz and HIV-1 strains for the Vif, Nef, Vpr and gp41 deduced amino acid sequences are shown for selected regions of the proteins. Sequences are compared to SIVcpzTAN1, with dashes denoting sequence identity and dots representing gaps to optimize sequence alignment. Question marks indicate sites of ambiguous sequence in SIVcpz or sites where fewer than 50% of the viruses contain the same amino acid residue (in HIV-1). HIV-1 group M, N and O consensus sequences were obtained from the Los Alamos HIV sequence database (http://hiv-web,lanl,gov). Vertical boxes represent SIVcpz lineage specific protein sequences in Vif, Vpr, Nef and gp41. Arrows denote a pair of conserved cysteine residues in the ectodomain of gp41 that is unique to P. t. schweinfurthii viruses (the horizontal line denotes the immunodominant region of the HIV-1 gp41 glycoprotein). Asterisks indicate the highly conserved PPLP motif in Vif, a diacidic β-COP motif in Nef and four C-terminal Arg residues in Vpr (Arg 90 is circled).

FIG. 5 shows a phylogenetic tree of a SIVcpzTAN2 Env/Nef amino acid sequence and other SIVcpz and HIV-1 strains.

### DETAILED DESCRIPTION

The present disclosure relates to the determination of the complete genomic nucleic acid sequence of a new simian immunodeficiency virus (SIVcpzTAN1) isolated from a wild chimpanzee (Ch-06) from Gombe National Park in Tanzania and to the nucleic acids derived therefrom. Chimpanzee Ch-06 was a healthy, 24 year old, sexual active, mid-ranking male member of the Kasekela community in Gombe National Park. This community comprises approximately 55 members. All members of the community live freely (94). The disclosure also relates to the peptides encoded by and/or derived from the SIVcpzTAN1 nucleic acid sequence, to host cells containing the nucleic acids sequences and/or peptides, to diagnostic kits, immunogens and methods which employ the nucleic acids, peptides and/or host cells of the present disclosure, and to non-invasive methods for the detection of SIV and related viruses from animal species in the wild. The complete nucleotide sequence of the SIVcpzTAN1 is disclosed in SEQ ID NO: 1. The nucleotide sequence is in the R-U5-gag-pol-env-U3-R configuration and can be accessed through GENBANK (accession No. AF447763, which disclosure is incorporated by reference herein). The complete nucleotide sequence was amplified in overlapping fragments and sequenced and found to represent the entire genome. A replication competent SIVcpzTAN1 virus is not currently available. However, the applicants are in the process of constructing a replication competent SIVcpzTAN1 (represented by SEQ ID NO: 1) virus by combining the overlapping fragments. Such a procedure is within the ordinary skill of one in the art. When a replication competent SIVcpzTAN1 virus is obtained, a deposit will be made with the American Type Culture Collection (Manassas, Va.) or other International Depository Authority at which time information sufficient to identify and obtain the SIVcpzTAN1 virus will be added to this application.

The amino acid sequences of the polypeptides encoded by SEQ ID NO: 1 have also been deduced. The deduced amino acid sequence of the Gag, Pol, Vif, Vpr, Tat, Rev, Vpu, Env and Nef polypeptides are disclosed in SEQ ID NOS. 2-10, respectively.

As used throughout this disclosure, the term SIVcpzTAN1 nucleic acid (SEQ ID NO: 1) will refer to the nucleotide sequence of the new simian immunodeficiency virus derived from a wild chimpanzee (Ch-06) from Gombe National Park in Tanzania, and to related SIVcpz strains as well. By related SIVcpz strains, it is meant those SIVcpz strains that differ from SIVcpzTAN1 in their DNA sequence by less than or equal to 30%, or in other words have a percent homology of 70%, or that hybridize to all, or a portion of SEQ ID NO: 1, or the complement thereof, under stringent conditions. As used in this disclosure, the term “percent homology” of two amino acid sequences or of two nucleic acid sequences is determined using the algorithm of Karlin and Altschul, modified as in Karlin and Altschul (105). Such an algorithm is incorporated into the NBLAST and XBLAST programs of Altschul et al. (106). Blast nucleotide searches are performed with the NBLAST program, score=100, wordlength=12, to obtain nucleotide sequences homologous to a nucleic acid molecule of the invention. Blast protein searches are performed with the XBLAST program, score=50, wordlength=3 to obtain amino acid sequences homologous to a referenced polypeptide. To obtain gapped alignments for comparison purposes, Gapped BLAST is utilized as described in Altschul et al. (107). When utilizing BLAST and Gapped BLAST programs, the default parameters of the respective programs (XBLAST and NBLAST) are used. See http://www.ncbi.nlm.nih.gov.

The hybridizing portion of the hybridizing nucleic acid is generally 15-50 nucleotides in length. The hybridizing portion of the hybridizing nucleic acid is at least 50% to 98% identical to the sequence of at least a portion of the nucleotide sequence represented by SEQ ID NO: 1, or its complement. Hybridizing nucleic acids as described herein can be used for many purposes, such as, but not limited to, a cloning probe, a primer for PCR and other reactions, and a diagnostic probe. Hybridization of the hybridizing nucleic acid is typically performed under stringent conditions. Nucleic acid duplex or hybrid stability is expressed as the melting temperature Tm, which is the temperature at which the hybridizing nucleic acid disassociates with the target nucleic acid. This melting temperature is many times used to define the required stringency conditions. If sequences are to be identified that are related to and/or substantially identical to the nucleic acid sequence represented by SEQ ID NO: 1, rather than identical, then it is useful to establish the lowest temperature at which only homologous hybridization occurs with a particular concentration of salt (such as SSC or SSPE).

Assuming that 1% mismatch results in a 1° C. decrease in Tm, the temperature of the final wash in the hybridization reaction is reduced accordingly (for example, if a sequence having a 90% identity with the probe are sought, then the final wash temperature is decreased by 5° C. The change in Tm can be between 0.5° C. and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. The parameters of salt concentration and temperature can be varied to achieve the optimal level of identity between the probe and the target nucleic acid. Additional guidance regarding such conditions is readily available in the art.

The methods and techniques, as well as the uses for the SIVcpzTAN1 nucleic acid sequences and nucleic acid sequences derived therefrom and the polypeptides encoded by or derived from the nucleic acid sequences, would be applicable to the related SIVcpz strains as well.

One such related SIVcpz strain is SIVcpzTAN2. SIVcpzTAN2 was isolated from a chimpanzee termed GM-39 also from Gombe National Park in Tanzania. The chimpanzee from which SIVcpzTAN1 is derived (Ch-06) and the chimpanzee from which SIVcpzTAN2 is derived are living in different communities within Gombe National Park. The nucleotide sequence of several fragments from SIVcpzTAN2 have been isolated and sequenced. A 688 base pair fragment encompassing portions of the env and nef genes of SIVcpzTAN2 is disclosed in SEQ ID NO: 15 and the corresponding amino acid sequence of the Env and Nef polypeptide fragment is disclosed in SEQ ID NO: 16. In addition, a fragment encompassing a portion of the pol gene is disclosed in SEQ ID NO: 17 and the corresponding amino acid sequence of the Pol polypeptide fragment is disclosed in SEQ ID NO: 18.

Genomic Sequence of SIVcpzTAN1

The present disclosure relates to the determination of the nucleic acid sequence of the complete genome of SIVcpzTAN1 (SEQ ID NO: 1) and nucleic acids derivatives thereof. The term derivatives include the “fragments,” “variants,” “complementary sequences,” “degenerate variants” and “chemical derivatives.” The term “fragment” is meant to refer to any nucleic acid subset of SEQ ID NO: 1 incorporating or encoding 9 or more contiguous or sequential nucleic acid residues. The term “chemical derivative” describes an embodiment of SEQ ID NO: 1 that contains additional chemical moieties or domains, or altered levels of chemical moieties of domains, than are normally a part of the SEQ ID NO: 1.

It is known that there is a substantial amount of redundancy in the codons which code for specific amino acids. Therefore, this disclosure is directed to those nucleic acid sequences which contain alternative codons which code for the eventual translation of the identical amino acid specified in SEQ ID NO: 1. For purposes of this specification, a sequence bearing one or more alternative codons will be defined as a “degenerate variation.” Also included within the scope of this disclosure are mutations either in the nucleic acid sequence, and therefore the translated protein, which do not substantially alter the ultimate physical properties of the proteins encoded by SEQ ID NO: 1 and derivatives thereof, such as, but not limited to, the presence of conservative amino acid substitutions (defined in this specification as a “variant”). For the purpose of this specification, conservative amino acid substitutions include any substitutions within the groups of amino acids as defined in Zubay, Biochemistry, 2cd edition, p. 32, Macmillian Publishing Company, New York, N.Y. For example, conservative amino acid changes, such as, but not limited to, substitution of valine for leucine (Group I), asparagine for glutamine (Group II) or aspartic acid for glutamic acid (Group III).

A description of the amplification and compilation of SEQ ID NO: 1 is described in reference 94 (which reference is incorporated in its entirety as if fully set forth herein). The phrase derivative thereof is also describes nucleic acid sequences which correspond to a region of the designated nucleic acid sequence. The sequence of the region from which the nucleic acid is derived, or is complementary to, may be a sequence which is unique to the SIVcpzTAN1 genome. Whether or not a sequence is unique to the SIVcpzTAN1 genome can be determined by techniques well known in the art, including, but not limited to, GENBANK comparisons and hybridization techniques. Regions of the SIVcpzTAN1 genome from which nucleic acid sequences may be derived include, but are not limited to, regions encoding specific polypeptides and/or epitopes (such as those shown in SEQ ID NOS: 19-21), as well as non-translated or non-transcribed sequences. The epitope may be unique to the SIVcpzTan1 genome. The uniqueness of the epitope may be determined by its degree of immunological cross reactivity with other SIVs and or HIVs and through computer searches as described.

The SIVcpzTAN1 nucleic acid is not necessarily physically derived from the nucleic acid sequence disclosed in SEQ ID NO: 1, but may be generated in any manner based on the information provided in the sequence of bases in the region from which the nucleic acid is derived, including, but not limited to, chemical synthesis. The derived nucleic acid may be of any length, but preferably is comprised of at least 6-12 bases, more preferably 15-19 bases, more preferably 30 bases. In addition, regions or combinations of regions corresponding to that of the designated sequence may be modified in ways known in the art to be consistent with an intended use. The derived nucleic acid may be a polynucleotide or a polynucleotide analog.

The term recombinant nucleotide or recombinant nucleic acid as used herein intends a nucleic acid of genomic, cDNA, semi-synthetic or synthetic origin which by virtue of its origin or manipulation: 1) is not associated with all or a portion of the nucleic acid with which it is associated in nature; and/or 2) is linked to a nucleic acid other than to which it is linked in nature. The term polynucleotide as used herein refers to a polymeric form of nucleotides of any length, either ribonucleotides or deoxyribonucleotides. This term includes double- and single-stranded DNA, as well as double- and single-stranded RNA. It also includes modifications, such as, but not limited to, methylation and/or capping and unmodified forms of the polynucleotide.

Fragments may be obtained by various methods well known in the art, including, but not limited to, restriction digestion, PCR amplification and direct synthesis. Fragments may be all or part of the genes encoding the Gag, Pol, Vif, Vpr, Tat, Rev, Vpu, Env, and Nef polypeptides and or complementary sequences thereof. Nucleic acids also include cDNA, mRNA and other nucleic acids derived from the SIVcpzTAN1 genome.

The disclosure also includes the amino acid sequences of the proteins encoded by SEQ ID NO: 1. The deduced amino acid sequences of the Gag, Pol, Vif, Vpr, Tat, Rev, Vpu, Env, and Nef polypeptides are given in SEQ ID NOS. 2-10, respectively. Inspection of the deduced protein sequences from SEQ ID NO: 1 revealed the expected open reading frames for gag, pol, vif, vpr, vpu, tat, rev, env and nef genes. None of these open reading frames contained inactivating mutations. Furthermore, the major regulatory sequences, including promoter and enhancer elements in the LTR, the transactivating region stem-loop structure, the packaging signal, the primer binding site and the major splice sites all appeared to be intact. The nucleic acids described herein may be present in vectors or host cells, or can be isolated and substantially purified as taught by methods well known in the art.

Methods for Detecting SIVcpzTAN1 Related Viruses.

The present disclosure also relates to methods for detecting the presence of SIVcpzTAN1, and similar SIVcpz strains, in mammals. The nucleic acids, vectors comprising the nucleic acids of the disclosure and/or host cells comprising vectors comprising the nucleic acids of the disclosure can be used for this purpose. The nucleic acid sequences derived from SEQ ID NO: 1, or its complement, may be incorporated into a vector. Such a construction could be used for replicating said nucleic acid sequences in an organism or cell other than the natural host so as to provide sufficient quantities of said nucleic acids to be used for diagnostic purposes (such as the use of said nucleic acids as probes in diagnostic assays).

In one embodiment, the detection method involves analyzing DNA of a mammal suspected of harboring SIVcpzTAN1. The DNA of the mammal can be isolated using methods known in the art, and include, but are not limited to, Southern blotting (63), dot and slot hybridization (60) and nucleotide arrays (as described in U.S. Pat. Nos. 5,445,934 and 5,733,729). Nucleic acid probes specific to SIVcpzTAN1 may be used to detect the presence of SIVcpzTAN1 or related SIVcpz strains in said isolated DNA. The nucleic acid probes used in the detection methods mentioned above are derived from the nucleic acid sequence disclosed in SEQ ID NO: 1. The size of the probes can vary, but the probes are generally 10-12 bases long, but can be from 200 to over 1000 bases long. The selection of the appropriate probe and its composition is within the skill of one in the art and can be designed with reference to SEQ ID NO: 1.

The nucleic acid probes may be DNA or RNA and can be synthesized using any known method of nucleotide synthesis (45, 55, and 58), or the probes can be isolated fragments of naturally occurring or cloned nucleic acids. In addition, the probes may be synthesized using automated instruments. The probes may also be nucleotide analogs, such as nucleotides linked by phosphodiester, phosphorothiodiester, methylphosphonodiester or methylphosphonthiodiester moieties (67) and peptide nucleic acids (68). The probes can also be labeled using methods known in the art, such as radiactive labels, biotin, avidin, enzymes and fluorescent molecules (62).

The nucleic acid probes used in the detection methods set forth above are derived from sequences substantially homologous to the sequence disclosed in SEQ ID NO: 1, or its complementary sequence. By substantially homologous it is meant a high level of homology between the nucleic acid probe and the nucleic acid sequence disclosed in SEQ ID NO: 1, or its complementary sequence. Preferably, the level of homology is greater than or equal to 80%, with a preferred homology being greater than or equal to 95%. Although complete complementarity is not required, it is preferred that the probes are constructed so that complete complementarity exists between the nucleic acid probe and the region of SIVcpzTAN1 to be detected.

In another embodiment, the detection method comprises analyzing RNA for the presence of SIVcpzTAN1 or SIVcpzTAN1 related viruses. The RNA can be isolated by methods well known in the art and include Northern blotting (66), dot and slot hybridization, filter hybridization (57), RNase protection (62) and polymerase chain reaction (PCR) (65). In one embodiment, the PCR is reverse-transcription-PCR (RT-PCR) whereby RNA is reversed transcribed to a first strand cDNA using a nucleic acid primer or primers derived from the nucleic acid sequence disclosed in SEQ ID NO: 1. After the cDNA is synthesized, PCR amplification is carried out using pairs of primers designed to hybridize with the sequences in the SIVcpzTAN1 nucleic acid to permit amplification of the cDNA and subsequent detection of the amplified product. Optimization of the amplification reaction to obtain sufficiently specific hybridization to the SIVcpzTAN1 nucleic acid sequences is well within the skill in the art and may be achieved by adjusting the annealing temperature.

The amplification products of PCR can be detected either indirectly or directly. For direct detection of the amplification products, primer pairs may be labeled. Labels suitable for such methods are known in the art and include, but are not limited to, radioactive labels, biotin, avidin, enzymes and fluorescent molecules. Alternatively, the desired labels can be incorporated into the primer extension products during the amplification reaction in the form of one or more labeled dNTPs. The labeled amplified PCR products can also be detected by ethidium bromide staining and visualization under UV light. The labeled amplified PCR products can also be detected by direct sequencing of the PCR products or by binding to immobilized oligonucleotide arrays. Unlabeled amplification products can also be detected by hybridization with labeled nucleic acid probes in methods known to those of skill in the art such as dot or slot blot hybridization assays.

By way of example, any of the probes described above may be used in a method incorporating the following steps: 1) labeling of the probe generated as described above by the methods previously described; 2) bringing the probe into contact under stringent hybridization conditions with nucleic acid, once said nucleic acid has been rendered accessible to the probe (such as by isolation on a membrane); 3) washing the membrane with a buffer under circumstances in which stringent conditions are maintained; and 4) detecting the probe by a suitable technique depending on the label employed.

The probes described above may also be packaged into diagnostic kits and may include the ingredients for labeling and the material needed for the particular detection protocol in addition to the probes.

Production of SIVcpzTAN1 Polypeptides

The disclosure also relates to methods of using the nucleic acid sequence disclosed in SEQ ID NO: 1 to direct the production of polypeptides in vitro or in vivo. In one embodiment, a recombinant method of making a polypeptide according to the disclosure comprises; 1) preparing a nucleic acid, derived from SEQ ID NO: 1 or its complement, capable of directing a host cell to produce a polypeptide encoded by the SIVcpzTAN1 genome; 2) cloning the nucleic acid into a vector capable of being transferred into and replicated in the host cell, the vector containing the operational elements for expressing the nucleic acid if required; 3) transferring the vector comprising the nucleic acid and operational elements into a host cell capable of expressing the polypeptide; 4) growing the host cell under conditions appropriate for the expression of the polypeptide; and 5) harvesting the polypeptide.

The present disclosure also relates to non-recombinant methods of expressing the polypeptides and nucleic acids described herein. In addition to synthetic methods of polypeptide and nucleic acid production, the non-recombinant methods involve culturing the SIVcpzTAN1 in cell lines, such as uninfected human peripheral blood mononuclear cells, under conditions appropriate for the expression of the polypeptides and nucleic acids. The polypeptides and nucleic acids can then be purified by methods known in the art.

The vectors which can be used in the present disclosure include any vectors into which a nucleic acid sequence as described above can be inserted, along with any preferred or required operational elements, and which the vector can be transferred into a host cell and preferably replicated by the host cell. It is advantageous if the restriction sites of the vector are well documented and the vector contains operational elements preferred or required for transcription of the nucleic acid sequence. The operational elements referred to above generally comprise at least one promoter sequence capable of initiating transcription of the inserted nucleic acid sequence, at least one leader sequence, at least one terminator codon and/or termination signal, and any other necessary or preferred DNA sequence for appropriate transcription and translation of the inserted nucleic acid sequence. It is contemplated that the vector will also contain at least one origin of replication recognized by the host cell with at least one selectable marker.

Expression vectors that may be used are those which function in bacterial and/or eukaryotic cells. Examples of vectors which operate in eukaryotic cells include, but are not limited to, Venezuelan equine encephalitis virus vectors, simian virus vectors, vaccinia virus vectors, adenovirus vectors, herpes virus vectors, or vectors based on retroviruses, such as murine leukemia virus, or lentiviruses (76). The expression vectors can also be transfected into bacterial or eukaryotic cell systems. Eukaryotic cell systems include, but are not limited to, cell lines such as HeLa, COS-1, 293T, MRC-5 or CV-1 cells. Primary human cells, such as lymph node cells, macrophages, are also useful in this regard.

The expressed polypeptides may be detected by methods known in the art including, but not limited to, Western blotting, Coumassie blue staining, through the detection of the expression product of a reporter gene (i.e., luciferase) or through measurement of the activity of the expressed polypeptide. In another embodiment of the invention, the method comprises administering a composition comprising a vector, the vector further comprising a nucleic acid sequence disclosed in SEQ ID NO: 1 to direct the production of polypeptides in vivo.

The polypeptides of the present disclosure refer to one or more of the polypeptides encoded by the nucleic acid sequence disclosed in SEQ ID NO: 1, and derivatives of SEQ ID NO: 1. Polypeptides encoded by SEQ ID NO: 1 and derivatives thereof include, but are not limited to, those polypeptides having the amino acid sequence of which is disclosed in SEQ ID NOS: 2-10. The polypeptides which are derivatives of the nucleic acid sequence disclosed in SEQ ID NO: 1 include polypeptides encoded by nucleic acids such as, but not limited to, degenerate variants, variants, chemical derivatives and fragments (as defined in this specification). The present disclosure also includes chemical derivatives of the polypeptides discussed above. The term “chemical derivative” is meant to refer to a polypeptide that contains additional chemical moieties or domains, or altered levels of chemical moieties or domains, than are normally associated with the polypeptide. Chemical derivatives include, but are not limited to, polypeptides having altered levels of glycosylation.

The polypeptides disclosed in SEQ ID NOS: 2-10 may be used as compositions comprising a pharmaceutically acceptable carrier either alone, in combination with one another, or in combination with other proteins of the lentivirus family, including but not limited to, other SIVs or HIVs. These polypeptides may be produced by synthetic or recombinant methods, or can be harvested from cells infected by SIVcpzTAN1. These polypeptides may be obtained and used as crude lysates or can be purified by standard protein purification techniques. These techniques include, but are not limited to, differential precipitation, molecular sieve chromatography, ion exchange chromatography, isoelectric focusing, gel electrophoresis and affinity and immunoaffinity chromatography. The polypeptides may be purified by passage through a column containing a resin which comprises bound antibodies specific for a given expressed epitope of an expressed polypeptide.

A polypeptide or amino acid sequence derived from a designated nucleic acid sequence refers to a polypeptide having an amino acid sequence identical to that of a polypeptide encoded by the sequence, or a portion thereof, where the portion may be of any length, but preferably comprises at least 6-8 amino acids, or at least 10 amino acids, or at least 11-15 amino acids or at least 30 amino acids, or which polypeptide is immunologically cross-reactive with a polypeptide derived from a designated nucleic acid sequence. Polypeptides from the V3-loop region and the crown of the polypeptide encoded by the nucleic acid sequences of the env gene may be particularly useful. The polypeptides of the present disclosure may be generated in any manner, including, but not limited to chemical synthesis, recombinant expression system, or isolation of the polypeptides from SIVcpzTAN1.

The nucleic acid disclosed in SEQ ID NO: 1 represents one embodiment of the present invention. Due to the degeneracy of the genetic code, it is understood that there are numerous choices of nucleotides that may give rise to a nucleic acid sequence capable of directing the production of the polypeptides discussed above and disclosed in SEQ ID NOS. 2-10. As such, nucleic acid sequences that are functionally equivalent to the sequence disclosed in SEQ ID NO: 1, such sequences are intended to be covered by the present disclosure. For example, the nucleic acid sequence disclosed in SEQ ID NO: 1 may be modified so that the sequence codes for the preferred codons which are appropriate for a host cell that is being used to express the polypeptides of the present disclosure. In addition, the nucleic acid sequence disclosed in SEQ ID NO: 1 may be modified to reduce the effect of any inhibitory sequences and/or any sequences that may lead to instability and/or to provide for rev-independent gene expression (77).

Use of SIVcpzTAN1 Polypeptides and Nucleic Acids as Immunogens

The polypeptides of the present disclosure can be used at an effective amount as immunogens to raise antibodies and/or stimulate cellular immunity in a mammal. The immunogen may be a partially or substantially purified polypeptide. Alternatively, the immunogen may be a cell or cell lysate from cells transfected with a recombinant expression vector comprising at least a portion of the nucleic acid disclosed in SEQ ID NO: 1 or derived from SEQ ID NO: 1, or a culture supernatant containing at least one polypeptide as disclosed in SEQ ID NOS. 2-10, or polypeptides derived from SEQ ID NOS. 2-10. The immunogen may comprise one or more structural proteins, and/or one or more non-structural proteins of SIVcpzTAN1, or a mixture thereof. For the purposes of the present invention, “mammal” as used throughout the specification and claims, includes, but is not limited to humans, chimpanzees, other primates and the like.

The effective amount of polypeptide of the present disclosure per unit dose sufficient to act as an immunogen (i.e., to induce an immune response depends), among other things, on the species of mammal inoculated, the body weight of the mammal and the chosen inoculation regimen, as well as the presence or absence of an adjuvant, as is well known in the art. Inocula typically contain polypeptide concentrations from about 1 microgram to about 50 milligrams per inoculation (dose), from about 10 micrograms to about 10 milligrams per dose, or from about 100 micrograms to about 5 milligrams per dose.

The term “unit dose” as it pertains to the inocula refers to physically discrete units suitable as unitary dosages for mammals, each unit containing a predetermined quantity of active material (such as polypeptide(s) of the present disclosure) calculated to produce the desired immunogenic effect in association with the required diluent. Inocula are typically prepared as a solution in a physiologically acceptable carrier such as saline, phosphate-buffered saline and the like to form an aqueous pharmaceutical composition. The route of inoculation is typically parenteral or intramuscular, sub-cutaneous and the like. The dose is administered at least once. In order to increase the antibody level, at least one booster dose may be administered after the initial injection, at about 4 to 6 weeks after the first dose. Subsequent doses may be administered as indicated.

To monitor the antibody response of individuals, antibody titers may be determined. In most instances it will be sufficient to assess the antibody titer in serum or plasma obtained from such an individual. Decisions as to whether to administer booster inoculations or to change the amount of the immunogen administered to the individual may be at least partially based on the titer. The titer may be based on an immunobinding assay which measures the concentration of antibodies in the serum which bind to a specific antigen. The ability to neutralize in vitro and in vivo biological effects of SIVcpzTAN1 may also be assessed to determine the effectiveness of the immunization. Other methods to determine the antibody titre may be used and are well known in the art.

For all therapeutic, prophylactic and diagnostic uses, the polypeptide of the present disclosure, alone or linked to a carrier, as well as antibodies and other necessary reagents and appropriate devices and accessories, may be provided in kit form so as to be readily available and easily used. Where immunoassays are involved, such kits may contain a solid support, such as a membrane (e.g., nitrocellulose), a bead, sphere, test tube, microtiter well and so forth, to which a receptor such as an antibody specific for the target molecule will bind. Such kits can also include a second receptor, such as a labeled antibody. Such kits can be used for sandwich assays. Kits for competitive assays are also envisioned.

In one embodiment, the polypeptides or nucleic acids of the present disclosure can be used to prepare antibodies against SIVcpzTAN1 epitopes that are useful in diagnosis and/or therapy and/or to stimulate the immune response. The term “antibodies” is used herein to refer to immunoglobulin molecules and immunologically active portions of immunoglobulin molecules. Exemplary antibody molecules are intact immunoglobulin molecules, substantially intact immunoglobulin molecules and portions of an immunoglobulin molecule, including those portions known in the art as Fab, Fab′, F(ab′)₂ and F(v) as well as chimeric antibody molecules.

An antibody of the present disclosure is typically produced by immunizing a mammal with an immunogen or vaccine. In one embodiment, the immunogen or vaccine contains one or more polypeptides of the present disclosure (SEQ ID NOS 2-10), or a structurally and/or antigenically related molecule from related SIVcpz strains, or other primate lentiviruses such as, but not limited to HIV-1, to induce, in the mammal, antibody molecules having immunospecificity for the immunizing polypeptide(s). The polypeptide(s) may be monomeric, polymeric, conjugated to a carrier, and/or administered in the presence of an adjuvant.

In another embodiment, the immunogen or vaccine contains one or more nucleic acids encoding one or more polypeptides of the invention, or one or more nucleic acids encoding structurally and/or antigenically related molecules, to induce, in the mammal, the production of the immunizing peptide(s). The antibody molecules may then be collected from the mammal if they are to be used in immunoassays or for providing passive immunity.

The antibodies produced as described above may be polyclonal or monoclonal. Monoclonal antibodies may be produced by methods known in the art. Portions of immunoglobulin molecules may also be produced by methods known in the art. The antibody of the present disclosure may be contained in various carriers or media, including blood, plasma, serum (e.g., fractionated or unfractionated serum), hybridoma supernatants and the like. Alternatively, antibodies may be isolated to the extent desired by well known techniques such as, for example, by using DEAF SEPHADEX, or affinity chromatography. The antibodies may be purified so as to obtain specific classes or subclasses of antibody such as IgM, IgG, IgA, IgG₁, IgG₂, IgG₃, IgG₄ and the like. Antibodies of the IgG class are useful for passive protection.

The presence of the antibodies of the present disclosure, either polyclonal or monoclonal, can be determined by, but are not limited to, the various immunoassays described above.

The antibodies produced by as described above have a number of diagnostic and therapeutic uses. The antibodies can be used as an in vitro diagnostic agents to test for the presence of SIVcpzTAN1 or SIVcpzTAN1 related viruses in biological samples in standard immunoassay protocols. The assays which use the antibodies to detect the presence of SIVcpzTAN1 or SIVcpzTAN1 related viruses in a sample involve contacting the sample with at least one of the antibodies under conditions which will allow the formation of an immunological complex between the antibody and the antigen that may be present in the sample. The formation of an immunological complex, if any, indicating the presence of SIVcpzTAN1 or SIVcpzTAN1 related viruses in the sample, is then detected and measured by suitable means. Such assays include, but are not limited to, radioimmunoassays (RIA), ELISA, indirect immunofluorescence assay, Western blot and the like. The antibodies may be labeled or unlabeled depending on the type of assay used. Labels which may be coupled to the antibodies include those known in the art and include, but are not limited to, enzymes, radionucleotides, fluorogenic and chromogenic substrates, cofactors, biotin/avidin, colloidal gold and magnetic particles. Modification of the antibodies allows for coupling by any known means to carrier proteins or peptides or to known supports, for example, polystyrene or polyvinyl microtiter plates, glass tubes or glass beads and chromatographic supports, such as paper, cellulose and cellulose derivatives, and silica.

Such assays may be, for example, of direct format (where the labeled first antibody reacts with the antigen), an indirect format (where a labeled second antibody reacts with the first antibody), a competitive format (such as the addition of a labeled antigen), or a sandwich format (where both labeled and unlabelled antibody are utilized), as well as other formats described in the art. In one such assay, the biological sample is contacted with antibodies of the present disclosure and a labeled second antibody is used to detect the presence of SIVcpzTAN1 related viruses, to which the antibodies are bound.

The antibodies produced as described above are also useful as a means of enhancing the immune response when administered at a therapeutically effective amount. The antibodies may be administered with a physiologically or pharmaceutically acceptable carrier or vehicle therefore. A physiologically acceptable carrier is one that does not cause an adverse physical reaction upon administration and one in which the antibodies are sufficiently soluble and retain their activity. The therapeutically effective amount and method of administration of the antibodies may vary based on the individual patient, the indication being treated and other criteria evident to one of ordinary skill in the art. A therapeutically effective amount of the antibodies is one sufficient to reduce the level of infection by one or more of the viruses of this disclosure or attenuate any dysfunction caused by viral infection without causing significant side effects such as non-specific T cell lysis or organ damage. The route(s) of administration useful in a particular application are apparent to one or ordinary skill in the art. Routes of administration of the antibodies include, but are not limited to, parenteral, and direct injection into an affected site. Parenteral routes of administration include but are not limited to intravenous, intramuscular, intraperitoneal and subcutaneous.

The present disclosure includes compositions of the antibodies described above, suitable for parenteral administration including, but not limited to, pharmaceutically acceptable sterile isotonic solutions. Such solutions include, but are not limited to, saline and phosphate buffered saline for intravenous, intramuscular, intraperitoneal, or subcutaneous injection, or direct injection into an area. Antibodies for use to elicit passive immunity in humans may be obtained from other humans previously inoculated with pharmaceutical compositions comprising one or more of the polypeptides of the disclosure. Alternatively, antibodies derived from other species may also be used. Such antibodies used in therapeutics suffer from several drawbacks such as a limited half-life and propensity to elicit an immune response. Several methods are available to overcome these drawbacks. Antibodies made by these methods are encompassed by the present disclosure and are included herein. One such method is the “humanizing” of non-human antibodies by cloning the gene segment encoding the antigen binding region of the antibody to the human gene segments encoding the remainder of the antibody. Only the binding region of the antibody is thus recognized as foreign and is much less likely to cause an immune response.

In providing the antibodies of the present disclosure to a recipient mammal, preferably a human, the dosage of administered antibodies will vary depending upon such factors as the mammal's age, weight, height, sex, general medical condition, previous medical history and the like. In general, it is desirable to provide the recipient with a dosage of antibodies which is in the range of from about 5 mg/kg to about 20 mg/kg body weight of the mammal, although a lower or higher dose may be administered. In general, the antibodies will be administered intravenously (IV) or intramuscularly (IM).

The immunogens of this disclosure can also be generated by the direct administration of nucleic acids of this disclosure to a subject. DNA-based vaccination has been shown to stimulate humoral and cellular responses to HIV-1 antigens in mice (69-72) and macaques (72, 73). A DNA-based vaccine containing HIV-1 env and rev genes was injected into HIV infected human patients in three doses (30, 100 or 300 micrograms) at 10-week intervals. Increased antibodies against gp120 were observed in the 100 and 300 μg groups. Increases were also noted in cytotoxic T lymphocyte (CTL) activity against gp160-bearing targets and in lymphocyte proliferative activity (78, 79). DNA-based vaccines containing HIV gag genes, with modification of the viral nucleotide sequence to incorporate host-preferred codons (WO 98/34640), and/or to reduce the effect of inhibitory/instability sequences (77), have likewise been described.

Therefore, it is anticipated that the direct injection of RNA or DNA vectors of this disclosure encoding viral antigen can be used for endogenous expression of the antigen to generate the viral antigen for presentation to the immune system without the need for self-replicating agents or adjuvants, resulting in the generation of antigen-specific CTLs and protection from a subsequent challenge with a homologous or heterologous strain of SIVcpzTAN1. CTLs in both mice and humans are capable of recognizing epitopes derived from conserved internal viral proteins and are thought to be important in the immune response against viruses. By recognition of epitopes from conserved viral proteins, CTLs may provide cross-strain protection. CTLs specific for conserved viral antigens can respond to different strains of virus, in contrast to antibodies, which are generally strain-specific.

Thus, direct injection of RNA or DNA encoding the viral antigen has the advantage of being without some of the limitations of direct peptide delivery or viral vectors (81). Furthermore, the generation of high-titer antibodies to expressed proteins after injection of DNA indicates that this may be a facile and effective means of making antibody-based vaccines targeted towards conserved or non-conserved antigens, either separately or in combination with CTL vaccines targeted towards conserved antigens. These may also be used with traditional peptide vaccines, for the generation of combination vaccines. Furthermore, because protein expression is maintained after DNA injection, the persistence of B and T cell memory may be enhanced, thereby engendering long-lived humoral and cell-mediated immunity.

Nucleic acids encodingSIVcpzTAN1 polypeptides of this disclosure can be introduced into animals or humans in a physiologically or pharmaceutically acceptable carrier using one of several techniques such as injection of DNA directly into human tissues, electroporation or transfection of the DNA into primary human cells in culture (ex vivo), selection of cells for desired properties and reintroduction of such cells into the body, (said selection can be for the successful homologous recombination of the incoming DNA to an appropriate pre-selected genomic region); generation of infectious particles containing the SIVcpzTAN1 gag and/or other SIVcpzTAN1 genes, infection of cells ex vivo and reintroduction of such cells into the body, or direct infection by said particles in vivo. Substantial levels of polypeptide will be produced leading to an efficient stimulation of the immune system.

Also envisioned are therapies based upon vectors, such as viral vectors containing at least a portion of the nucleic acid sequences disclosed in or derived from SEQ ID NO: 1 and coding for the polypeptide(s) of the present disclosure. These vectors, developed so that they do not provoke a pathological effect, will stimulate the immune system to respond to the polypeptides expressed therefrom. The effective amount of nucleic acid or polypeptide immunogen per unit dose to induce an immune response depends, among other things, on the species of mammal inoculated, the body weight of the mammal, the chosen inoculation regimen and the use of an adjuvant as is well known in the art and described previously. Immunization can be conducted by conventional methods. For example, the immunogen can be used in a suitable diluent such as saline or water, or complete or incomplete adjuvants. Further, the immunogen may or may not be bound to a carrier. While it is possible for the immunogen to be administered in a pure or substantially pure form, it is preferable to present it as a pharmaceutical composition, formulation or preparation.

The formulations comprise an immunogen as described above, together with one or more pharmaceutically acceptable carriers and optionally other therapeutic ingredients. The carrier(s) must be “acceptable” in the sense of being compatible with the other ingredients of the formulation and not deleterious to the recipient thereof. The formulations may conveniently be presented in unit dosage form and may be prepared by any method well-known in the pharmaceutical art. The immunogen can be administered by any route appropriate for antibody production such as intravenous, intraperitoneal, intramuscular, subcutaneous, and the like. The immunogen may be administered once or at periodic intervals until a significant titer of antibody is produced. The antibody may be detected in the serum using an immunoassay. The host serum or plasma may be collected following an appropriate time interval to prove a composition comprising antibodies reactive with the SIVcpzTAN1 virus particles or encoded polypeptides. The gamma globulin fraction or the IgG antibodies can be obtained, for example, by use of saturated ammonium sulfate or DEAE Sephadex, or other techniques known to those skilled in the art.

In addition to its use to raise antibodies, the administration of the polypeptide and/or nucleic acid immunogens as described in the present disclosure may be for use as a vaccine for either a prophylactic or therapeutic purpose. When provided prophylactically, a vaccine(s) of the disclosure is provided in advance of any exposure to a SIVcpzTAN1 or SICcpzTAN1 related virus, such as HIV-1, or in advance of any symptoms due to such exposure. When provided therapeutically, a vaccine(s) of the disclosure is provided at (or shortly after) the onset of exposure to a SIVcpzTAN1 or SIVcpzTAN1 related virus, such as HIV-1, or at the onset of any symptom of infection or any disease or deleterious effects caused by such exposure. The therapeutic administration of the vaccine(s) serves to attenuate the infection or disease. The vaccine(s) of the present disclosure may, thus, be provided either prior to the anticipated exposure to a SIVcpzTAN1 or SIVcpzTAN1 related virus, such as HIV-1, or after the initiation of infection caused bys such exposure.

The use of polypeptides of the present disclosure is potentially advantageous for the use in vaccine preparations. It has been demonstrated that glycosylation plays a role in limiting the neutralizing antibody response to SIV and in shielding the virus from immune recognition (93). In addition, it has been shown that removing glycosylation sites from the env proteins of HIV-1 increases the level of neutralizing antibody to the env polypeptide. Table 1 shows a compilation of putative glycosylations sites, comparing SIVcpz with HIV-1 envelope amino acid sequences. Table 1 demonstrates that SIVcpz envelope glycoproteins, on average, have fewer glycosylation sites. When examining the known strains of SIVcpz, an average of 21.7 glycozylation sites are found per virion. This is compared to an average of 24.7 glycosylation sites per viorion for HIV-1 strains. Therefore, polypeptides encoded by or derived from SIVcpzTAN1 may make more effective immunogens for eliciting neutralizing antibodies in vaccine preparations.

While any of the polypeptides of the present disclosure or nucleic acids of the present disclosure can be used in vaccine preparation, for production of an optimal immune response, regions of conserved sequence identified in SIVcpzTAN1 as compared with other strains of SIV and HIV may be used. Identifying such conserved regions is well within the skill in the art and can be accomplished by computer searches and other well recognized methods. In this manner the immune response generated will be more likely to react with other strains of primate lentiviruses, including but not limited to SIVcpz strains and HIV-1. The polypeptides/nucleic acids of the present disclosure may be used alone or in combination with each other to generate the desired immune response. In addition, the polypeptides/nucleic acids of the present disclosure can be used in combination with other proteins derived from primate lentiviruses, including but not limited to, SIVcpz strains or HIV-1. In this manner the immune response and effectiveness of a vaccine preparation may be increased.

The disclosure also relates to the use of antisense nucleic acids to inhibit translation of peptides encoded by SIVcpzTAN1. The antisense nucleic acids are complementary to SIVcpzTAN1 mRNAs encoding peptides of this disclosure. The antisense nucleic acids may be in the form of synthetic nucleic acids or they may be encoded by a nucleotide construct, or they may be semi-synthetic. The antisense nucleic acids may be delivered to the cells using methods known to those skilled in the art.

Kits designed for diagnosis of SIVcpzTAN1 in a biological sample can be constructed by packaging the appropriate materials, including the nucleic acids and/or polypeptides of this disclosure and/or antibodies which specifically react with SIVcpzTAN1 antigens, along with other reagents and materials required for the particular assay.

Production of Diagnostic Reagents for SIVcpzTAN1 and Related Viruses

The disclosure also relates to any composition which can be use for the diagnosis of SIVcpzTAN1 infections or infections caused by SIVcpzTAN1 related viruses or for tests which have a prognostic value. These diagnostic procedures involve the detection of antibody in serum or other body fluid, which are directed against at least one of the antigens of SIVcpzTAN1.

In one embodiment, the compositions used to detected said antibodies comprise viral lysates or purified antigens which contain at least one of the viral core proteins or envelope proteins or pol gene derived proteins either alone or in various combinations. In an alternate embodiment, the composition used to detect said antibodies comprise either SIVcpzTAN1 viral lysate or polypeptides in combination with similarly prepared proteins derived from HIV-1 and/or HIV-2, and/or other SIVcpz strains such as SIVcpz-Gab and/or SIVcpzANT and/or SIVcpzCAM and/or related lentiviruses. This method may be used for the general diagnosis of infection or contact with immunodeficiency virus without regard to the absolute identity of the virus being detected.

Furthermore, the disclosure relates to a polypeptide(s) encoded by or derived from SEQ ID NO: 1 comprising an epitope that is recognized by serum of individuals carrying anti-SIVcpzTAN1 antibodies, or antibodies against SIVcpzTAN1 related viruses. The amino acid sequences corresponding to these epitopes can readily be determined by isolating the individual polypeptides, or fragments thereof, either by preparative electrophoresis or by affinity chromatography and determining the amino acid sequences of either the entire protein or the fragments produced enzymatically by trypsin or chymotrypsin digestion or by chemical means. The resulting peptide or polypeptides can subsequently be sequenced. The disclosure relates therefore to expressing any polypeptide comprising an epitope as discussed above, either derived directly from SIVcpzTAN1, or produced by synthetic or recombinant methods based on or derived from the nucleic acid sequence disclosed in SEQ ID NO: 1, and purifying the expressed protein. In particular, the disclosure relates to epitopes contained in any of the SIVcpzTAN1 core proteins, or in a protein which may contain a as part of its polypeptide chain epitopes derived from a combination of the core proteins. Furthermore, the invention relates to epitopes contained in either of the two SIVcpzTAN1 envelope glycoproteins, as well as any protein which contains, as part of its polypeptide chain, epitopes derived from a combination of the SIVcpzTAN1 envelope glycoprotein or a combination of the SIVcpzTAN1 core protein.

Furthermore, the disclosure relates to methods for the detection of antibodies against SIVcpzTAN1 in a biological fluid, in particular for the diagnosis of a potential or existing AIDS Related Complex or AIDS caused by SIVcpzTAN1, characterized by contacting body fluid of a person to be diagnosed with a composition containing one or more of the polypeptide encoded by or derived from SEQ ID NO: 1 or with a lysate of the virus, or with a polypeptide possessing epitopes common to SIVcpzTAN1, and detecting the immunological conjugate formed between the SIVcpzTAN1 antibodies and the antigen(s) used. Preferred methods include, but are not limited to, immunofluorescence assays or immunoenzymatic assays (61), radioimmunoassays, chemiluminescent assays, immunohistochemical assays and Western blot assays. Immunofluorescence assays typically involve incubating, for example, serum from the person to be tested with cells infected with SIVcpzTAN1 and which have been fixed and permeabilized with cold acetone. Immune complexes formed are detected using either direct or indirect methods and involve the use of antibodies which specifically react to human immunoglobulins. Detection is achieved by using antibodies to which have been coupled fluorescent labels, such as fluorescein or rhodamine.

Any of the polypeptides discussed above may be prepared in the form of a kit, alone, or in combination with other reagents such as secondary antibodies, for use in immunoassays.

The following examples illustrate certain embodiments of the present disclosure, but should not be construed as limiting its scope in any way. Certain modifications and variations will be apparent to those skilled in the art from the teachings of the forgoing disclosure and the following examples, and these are intended to be encompassed by the spirit and scope of the disclosure. The references disclosed herein, including United States and foreign patents and/or patent applications, are hereby incorporated by reference into this application.

### EXAMPLE 1

Detection of SIVcpz in Wild Chimpanzees.

Sampling blood from endangered primates is neither generally feasible or ethical. Non-invasive methods are described to detect and characterize SIVcpz in wild chimpanzees by analyzing fecal and urine samples for SIVcpz antibodies and virion RNA (83, 94). Urine samples (1-3 ml) and fecal samples (20-50 g) were collected from captive or wild chimpanzees under direct observation and stored at −20° C. Some fecal samples were preserved in RNAlater (Ambion, Austin, Tex.) to allow for storage and shipment at room temperature (see reference 94 regarding collection of samples and RNA purification from samples).

In order to determine which chimpanzees may be infected with a SIVcpz strain, Western Blot analysis and diagnostic PCR were conducted. For Western Blotting, HIV-1 nitrocellulose strips (Calypte Biomedical, Rockville, Md.) were blocked with 5% skim milk and incubated overnight at 4° C. with either 1 ml of undiluted urine or 1 ml of clarified fecal extracts in immunoblot buffer (PBS, pH 7.4, 5 mM EDTA, 0.05% Tween-20, 0.15 mM NaN₃, 1% BSA and 0.01% IGEPAL detergent). The strips were then reacted for one hour at room temperature with goat anti-human IgG (1:4000) conjugated to horseradish peroxidase and developed using an enhanced chemiluminescence detection system (Amersham/Pharmacia Biotech, Piscataway, N.J.). Immunoblots reactive with the HIV-1 envelope glycoprotein gp160 alone or in combination with other viral bands, or with any of the three structural proteins exclusive of gp16, were scored as positive. The absence of viral bands was scored negative, and samples not meeting either criterion were scored indeterminate. None of the urine or fecal samples tested exhibited indeterminate banding patterns.

RNA was analyzed by extraction from fecal samples using the RNAqueous Midi kit (Ambion, Austin, Tex.) (94). The RNA was analyzed using diagnostic PCR. Following cDNA synthesis, diagnostic PCR was performed using primers F1/R1, (SEQ ID NOS. 11 and 12, respectively) and F2/R2 (SEQ ID NOS. 13 and 14, respectively) Extension fragments of SIVcpzTAN1 were obtained using SIVcpzTAN1 specific primers and consensus primers.

The sensitivity and specificity of the antibody and RNA detection (via PCR) methods were tested in captive chimpanzees of known HIV or SIVcpz status (83). The sensitivity of the antibody detection was 100% for urine and 65% for feces. The specificity in each case was 100%. The sensitivity of the RNA detection from feces was 66%. The probabilistic methods used are described in reference 83.

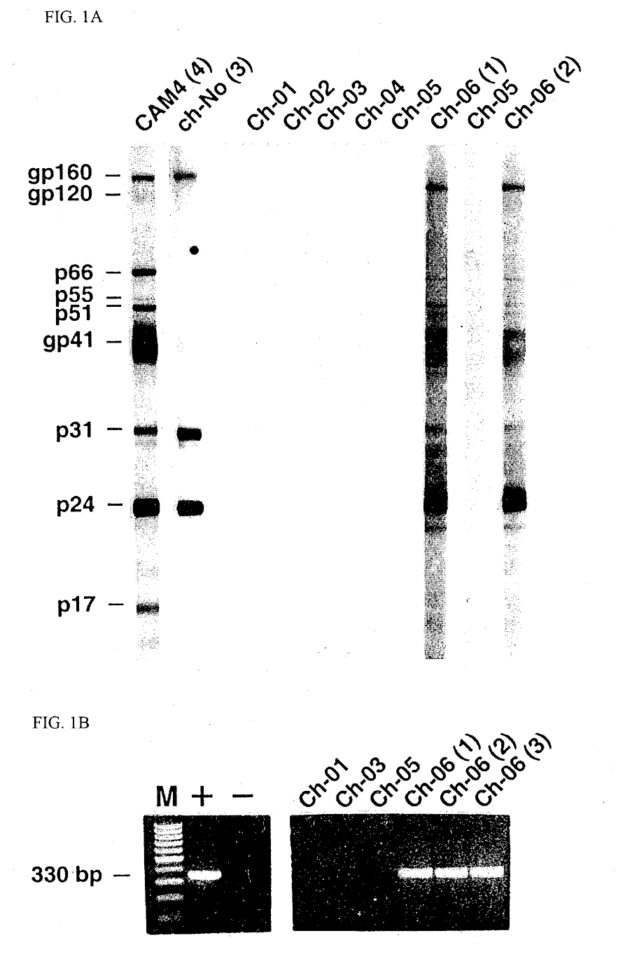

Using the techniques described, in an initial survey 58 wild-living chimpanzees were tested for the presence of SIVcpz. Of the 58 chimpanzees tested, 28 were P. t. verus from Tai Forest, Cote d'Ivoire, 24 were P. t. schweinfurthii from Kibale National Park, Uganda, 6 were P. t. schweinfurthii from Gombe National Park, Tanzania. Only one chimpanzee (designated Ch-06) tested positive for SIVcpz infection. Two different urine samples contained SIVcpz virion antibodies (FIG. 1A) and three fecal samples were positive for SIVcpz virion RNA (FIG. 1B). The full length sequence was subsequently derived by PCR amplification of overlapping subgenomic fragments (83, 94). Since this initial survey we have screened additional chimpanzees from Gombe which led to the identification of GM-39 to be infected with SIVcpzTAN2

### EXAMPLE 2

Comparison of SIVcpzTAN1 to Other SIVcpz and HIV Strains

The 2,195 bp pol/vif fragment amplified from fecal samples was initially sequenced and the amino acid sequence encoded by this fragment deduced and compared to comparable amino acid sequences from other SIVcpz and HIV strains. The results indicated SIVcpzTAN1 was a highly divergent SIVcpz strain. SIVcpzTAN1 differed from west-central African SIVcpz strains and HIV-1 groups M, N, and O by 28% and 30% of amino acid sequence (83, 94). The most similar sequence was that from SIVcpzANT (which was taken from a captive P. t. schweinfurthii of unknown origin) which differed from the amino acid sequence of SIVcpzTAN1 by 23% (83, 94).

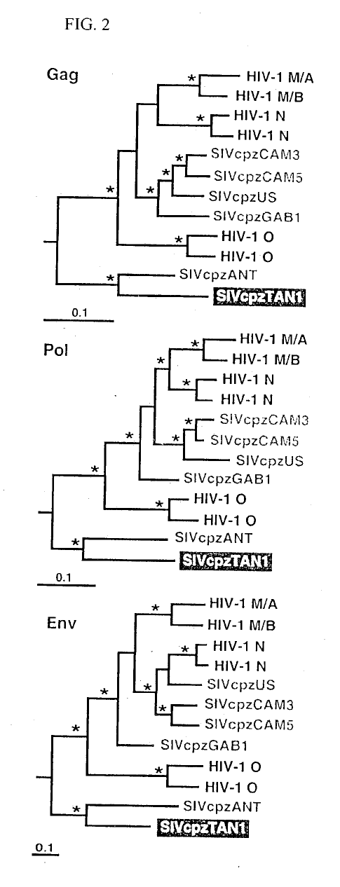

This was confirmed when the full length amino acid sequences of the SIVcpzTAN1 Gag, Pol and Env polypeptides were compared to other SIVcpz and HIV-1 strains. The phylogenetic tree shown in FIG. 2 demonstrates that SIVcpzTAN1 and SIVcpzANT cluster together in a highly significant manner, demonstrating that SIVcpzTAN1 fell within the HIV-1/SIVcpz radiation and grouped most closely with SIVcpzANT. This phylogenetic position was consistent in all major coding regions and supported by significant bootstrap values (FIG. 2). Distance and phylogenetic analyses thus identified SIVcpzTAN1 as a highly divergent member of the HIV-1/SIVcpz group of viruses. Since, until now, there has only been a single divergent P. t. schweinfurthii strain from a captive chimpanzee (Noah) of unknown origin, the possibility existed that SIVcpzANT was the result of a cross-species transmission event from another primate species and did not really represent a virus naturally infecting chimpanzees. The derivation of the complete SIVcpzTAN1 sequence from a chimpanzee of unquestionable provenance renders this possibility improbable. The phylogenetic position of TAN1 (shown in FIG. 2) confirms the authenticity of SIVcpzANT as a bona-fide SIVcpz strain and thus provides conclusive evidence for the existence of two major lineages within the SIVcpz/HIV-1 radiation.

### EXAMPLE 3

Vpu Amino Acid Sequence from SIVcpzTAN1 is Highly Divergent From Other SIVcpz and HIV-1 Strains

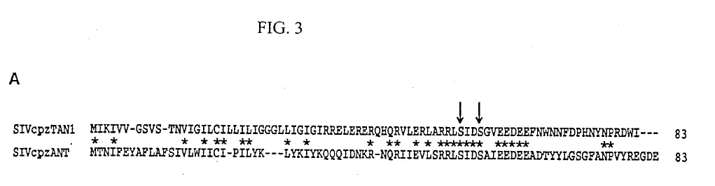

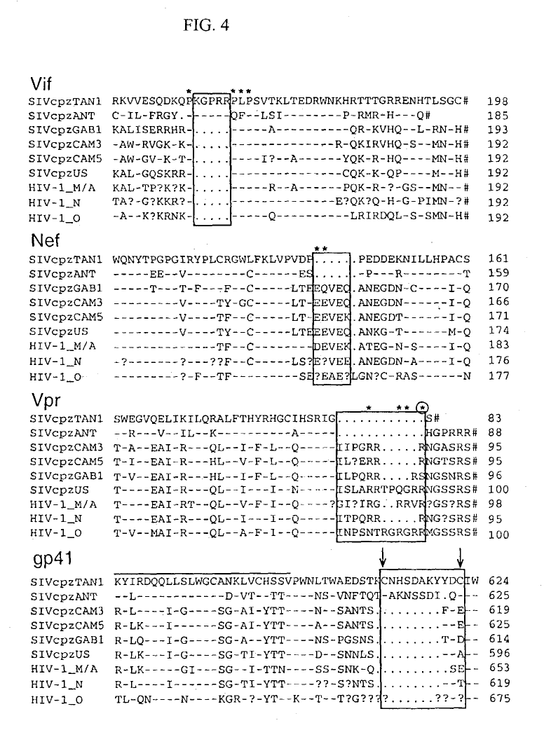

The deduced amino acid sequence of the Vpu protein (SEQ ID NO: 8) is highly divergent from other SIVcpz and HIV-1 proteins (FIG. 3). The TAN1 and ANT Vpu proteins were only 37% identical. However, the position of the vpu open reading frame and the overall hydrophobicity profile of the deduced protein sequence were very similar to other SIVcpz and HIV-1 strains, suggesting that the Vpu protein in SIVcpzTAN1 is functional. In addition, secondary structure predictions suggested the presence of alpha helices near the C-terminus that flanked two highly conserved serine residues (FIG. 3) previously shown to be critical for HIV-1 Vpu mediated CD4 degradation (95). Together, these data suggest that TAN1 encodes a functional Vpu protein.

### EXAMPLE 4

SIVcpzTAN1 Contains Several SIVcpz Signature Motifs

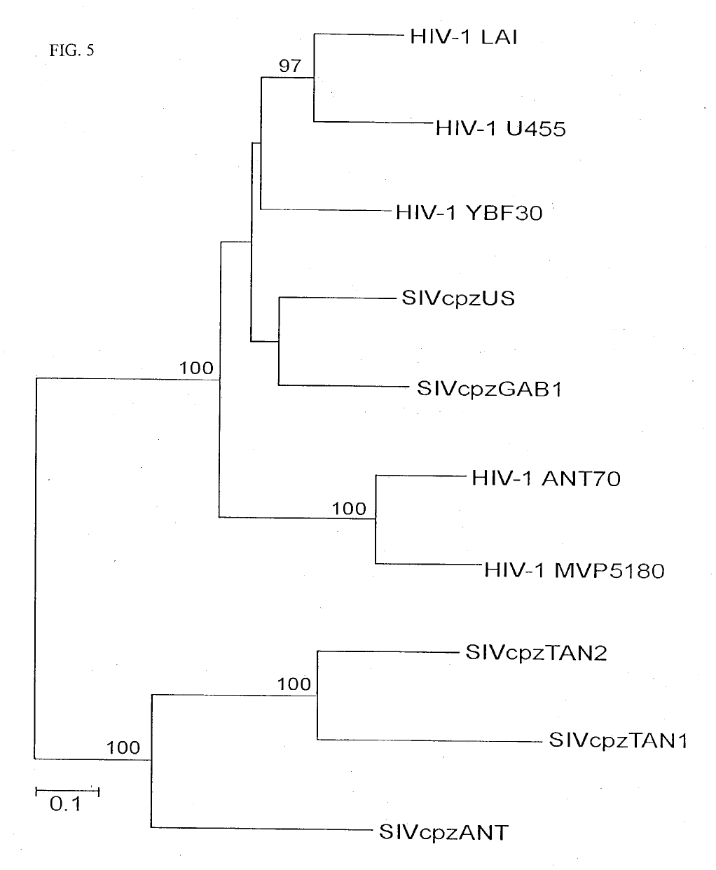

Analysis for lineage specific amino acid sequence insertions and deletions identified several signatures that distinguished ANT and TAN1 from all other SIVcpz and HIV-1 strains (FIG. 5). These lineage specific amino acid sequences may provide a mechanism to specifically screen for and/or detect the presence of the TAN1/ANT lineage in the SIVcpz/HIV-1 radiation. In one embodiment, the conserved signature motifs are used to generate specific probes to detect the presence of TAN1/ANT lineage nucleic acid in a sample. In another embodiment, the conserved signature motifs may be used to generate antibodies to detect the presence of TAN1/ANT lineage polypeptides in a sample. In addition to generating diagnostic reagents, the conserved signature motifs may be used for therapeutic purposes, such as in the development of vaccines specific to the TAN1/ANT lineage, or to stimulate the an immune response in a subject, such as a human. In one embodiment, the conserved sequence motif is selected from the group consisting of SEQ ID NOS. 19-21. In an alternate embodiment, the conserved sequence motif is SEQ ID NO: 20. In additions, the conserved signature motifs may be used as described in the instant specification.

TAN1 and ANT contained an identical five amino acid insertion (KGPRR) (SEQ ID NO: 19) near the C-terminus of Vif which disrupted a highly conserved PPLP motif previously shown to be critical, in its entirety, for HIV-1 Vif function (96). In addition, they exhibited a five amino acid deletion near the C-terminus of Nef that included a diacidic β-COP (coatomer protein) binding motif shown to be important for HIV-1 Nef induced CD4 degradation (97). Both ANT and TAN1 also encoded a considerably truncated Vpr protein that lacked several basic residues at the C-terminus previously shown to be important for HIV-1 Vpr induced nuclear localization and G2 cell cycle arrest, including a critical Arg-90 residue (98). Since accessory protein functions are highly conserved among divergent SIV lineages, it is highly unlikely that the Vif, Vpr, and Nef proteins of the two P. t. schweinfurthii viruses have lost these functions (this is especially true for TAN1 which was derived without the in vitro selection that might occur through growth in human T-cell lines). Instead, the observed Vif, Vpr and Nef mutations are likely compensated by amino acid substitutions elsewhere in these proteins. Finally, both ANT and TAN1 exhibited an amino acid sequence insertion (an 11 amino acids for TAN1 (SEQ ID NO: 20); and a 10 amino acids for ANT (SEQ ID NO: 21)) in the ectodomain of the transmembrane envelope glycoprotein (gp41) which is bounded by two additional cysteine residues (FIG. 5). Interestingly, although the motif is specific to the TAN1 and ANT SIVcpz strains, the amino acid of the sequences is not conserved between TAN1 and ANT. Unpaired cysteines are known to interfere with the proper folding of the SIV/HIV envelope glycoprotein (99-101). It is thus likely that the additional cysteine residues in TAN1 and ANT gp41 form intermolecular disulfide bonds, possibly resulting in an additional surface loop that might alter the local gp41 structure. Since this region is also known to be involved in gp120/gp41 interactions (102, 103), it is possible that compensatory changes in the N- or C-terminus of gp120 have evolved in association with these mutations. Interestingly, the extra cysteine pair in gp41, the truncated Vpr, and the Vif insertion were not only absent from SIVcpz from P. t. troglodytes but also from all other SIVs, including the relatively more closely related (at least in env) SIVgsn strain (104). This would suggest that P. t. schweinfurthii viruses have acquired these changes some time after their divergence from the common SIVcpz ancestor but before the split of the lineages represented by today's SIVcpzTAN1 and SIVcpzANT. In addition, the absence of these signatures from all known HIV-1 variants (groups M, N and O) is consistent with their west central African chimpanzee (P. t. troglodytes) origin.

### EXAMPLE 5

Comparison of SIVcpzTAN2 to Other SIVcpz and HIV Strains

The 688 bp sequence from SIVcpzTAN2 corresponding to a fragment of the env and nef genes is disclosed in SEQ ID NO: 15 and a 335 bp sequence corresponding to a fragment of the pol gene is disclosed in SEQ ID NO: 17. The amino acid sequence of the the env and nef gene fragment was deduced and is shown in SEQ ID NO: 16. The deduced amino acid sequence of the pol gene is shown in SEQ ID NO: 18. The amino acid sequences for the Env/Nef and Pol polypeptides were deduced and compared to corresponding amino acid sequences from other SIVcpz and HIV strains. SIVcpzTAN2 is 13% divergent from the corresponding amino acid sequence from SIVcpzTAN1. In the phylogenetic tree shown in FIG. 6, SIVcpzTAN2, SIVcpzTAN1 and SIVcpzANT clustered together in a highly significant manner. This indicates that SIVcpzTAN1, SIVcpzTAN2 and SIVcpzANT are highly divergent from HIV groups M, N, and O and further supports the conclusion that P. t. schweinfurthii did not serve as the zoonotic source for epidemic HIV.

### References

1. Allan, et al., 1991, J. Virol. 65:2816-2828.

2. Barre-Sinoussi, et al., 1983, Science 220:868-871.

3. Chen, Z., et al., 1997, J. Virol. 71:3953-3960.

4. Chen, Z., et al., 1996, J. Virol. 70:3617-3627.

5. Chen, Z., et al., 1995, J. Med. Primatol. 24:108-115.

6. Chen, Z et al., 1997, J. Virol. 71:2705-2714.

7. Clavel, F., et al., 1986, Science 233:343-346.

8. Daniel, M. D., et al., 1985, Science, 228:1201-1204.

9. Emau, P., et al., 1991, J. Virol. 65:2135-2140.

10. Faulkner, D. M. and J. Jurka. 1988, Science, 13:321-322.

11. Felsenstein, J. 1988, Annu. Rev. Genet. 22:521-565.

12. Felsenstein, J. 1989. PHYLIP—Phylogeny Inference Package (Version 3.2). Cladistics 5:164-166.

13. Fultz, P. N, et al., 1986, Proc. Natl. Acad Sci. USA 83:5286-5290.

14. Gao, F., et al., 1994, J. Virol. 68:7433-7447.

15. Gao, F., et al., 1992, Nature (London) 358:495-499.

16. Garnett, G. P., and R. Antia. 1994. Population Biology of Virus—Host Interactions. In The Evolutionary Biology of Viruses, Raven Press, New York, N.Y.

17. Grubb, L. 1982. Refuges and dispersal in the speciation of African forest mammals. In Biological Diversification in the Tropics, G. T. Prance (ed.) Columbia University Press, New York pp 537-553.

18. Hirsch, V. M., et al., 1989, Nature (London) 339:389-392.

19. Huet, T., et al., 1990, Nature (London) 345:356-359.

20. Janssens, W., 1994, AIDS Res. Human Retro. 10:1191-1192.

21. Jin, M. J., 1994, EMBOJ 13:2935-2947.

22. Johnson, P. R., et al., 1990, J. Virol. 64:1086-1092.

23. Kestler, H. W., et al., 1988, Nature (London) 331:619-622.

24. Kimura, M. 1983. The neutral theory of molecular evolution. Cambridge University Press, Cambridge, United Kingdom.

25. Kraus, G., et al., 1989, Proc. Natl. Acad. Sci. USA 86:2892-2896.

26. Kusumi, K., et al., 1992, J. Virol. 66:875-885.

27. Kwon, D., et al., Unpublished data.

28. Letvin, N. L., et al., 1985, Science 230:71-73.

29. Lowenstine, L. J., et al., 1986, Int. J. Cancer 38:563-574.

30. Marx, P. A., et al., 1993, Science 260:1323-1327.

31. Marx, P. A., et al., 1991, J. Virol. 65(8):4480-4485.

32. Marx, P. A., et al., 1996, Nature Medicine. Nature Medicine 2:1084-1089.

33. Miura, T., et al., 1990, AIDS 4:1257-1261.

34. Mojun J J, et al., 1994, EMBO J. 13:2935-2947.

35. Muller, M. C., et al., 1993, J. Virol. 67:1227-1235.

36. Murphey-Corb, M., et al., 1986, Nature (London) 321:435-437.

37. Myers, G., et al., 1995. Human retorviruses and AIDS. A compilation and analysis of nucleaic acid and amino acid sequences. Los Alamos National Laboratory, Los Alamos, N.M.

38. Myers, G., et al., 1992, AIDS Res. Hum. Retroviruses 8:373-386.

39. Nerienet E, et al., 1998, AIDS Res. Hum. Retroviruses, 14:785-96.

40. Ohta, Y., et al., 1988, Int. J. Cancer 41:115-122.

41. Otsyula, M., et al., 1996, Annals Trop. Med. Parisitol, 90:65-70.

42. Peeters, M., et al., 1992, AIDS 6:447-451.

43. Peeters, M., et al., 1989, AIDS 3:625-630.

44. Peeters, M., et al., 1994, AIDS Res. Hum. Retroviruses, 10:1289-1294.

45. Reimann, K. A., et al., 1994, J. Virol. 68:2362-2370.

46. Robbins C B. 1978, Bull. Carnegie Mus. Nat Hist. 6: 168-174.

47. Sharp, P. M., et al., 1994, AIDS 8 (Suppl.):S27-S42.

48. Stivahtis, G. L., et al., 1997, J. Virol. 71:4331-4338.

49. Stivahtis, G. L., et al., 1997, J. Virol. 71:4331-4338.

50. Tomonaga K, et al., 1993, Arch. Virol. 129:77-92.

51. Tsujimoto, H., et al., 1988, J. Virol. 62:4044-4050.

52. Vanden Haesevelde, M. M., et al., 1996, Virology 221:346-350.

53. Wolfheim, J. H. 1983. Primates of the world. Univ. of Washington, Seattle.

54. Agarwal et al. 1972, Angew. Chem. Int. Ed. Engl. 11:451. The phosphotriester method of Hsiung et al. 1979, Nucleic Acids Res. 6:1371.

55. Baeucage et al. 1981, Tetrahedron Letters 22:1859-1862. Automated diethylphosphoramidite method.

56. Biedleret et al. 1988. J. Immunol. 141:4053

57. Hollander, M. C. et al. 1990. Biotechniques; 9:174-179, RNase protection (Sambrook, J. et al. 1989. In “Molecular Cloning, a Laboratory Manual”, Cold Spring Harbor Press, Plainview, N.Y.).

58. Hsiung et al. 1979. Nucleic Acids Res 6:1371

59. Jones et al., 1986. Nature 321:552

60. Kafatos, F. C. et al. 1979. Nucleic Acids Res., 7:1541-1522

61. Oellerich, M. 1984. J. Clin. Chem. Clin. BioChem 22:895-904

62. Sambrook, J. et al. 1989. In “Molecular Cloning, A Laboratory Manual”, Cold Spring Harbor Press, Plainview, N.Y.

63. Southern, E. M. 1975. J. Mol. Biol., 98:503-517.

64. Verhoeyan, et al. 1988. Science 239:1534.

65. Watson, J. D., et al. 1992. In “Recombinant DNA” Second Edition, W. H. Freeman and Company, New York.

66. Alwine, J. C., et al. 1977. Proc. Natl. Acad. Sci., 74:5350-5354.

67. See, e.g., Anderson, et al. 1996. Antimicrob. Agents Chemother., 40:2004-2011; Azad, et al. 1995. Antiviral Res., 28:101-111; Azad, et al. 1993. Antimicrob. Agents Chemother., 37:1945-1954; Leeds, et al. 1997. Drug. Metab. Dispos., 25:921-926; and references therein. See also, Cook, P. D., 1993. Monomers for preparation of oligonucleotides having chiral phosphorus linkages. U.S. Pat. No. 5,212,295 (general method of making DNA analogs, including phosphorothioates, thioesters, etc.); and Iyer et al. 1990 J. Org. Chem. 55:4693-4699 (synthetic method for making phosphorothioate oligos).

68. See, e.g., Nielsen, et al., WO 98/03542; Hyrup and Nielsen 1996. Bioorg. Med. Chem. 4:5-23; and Nielsen, et al. 1991. Science 254:1497-1500; and references therein.

69. Lu S, et al., 1996, J. Virol., 70:3978-91.

70. Haynes J R, et al., 1994, AIDS Res Human Retroviruses, 10 (suppl 2): S43-5.

71. Okuda, K, et al., 1995, AIDS Res Hum Retroviruses, 11:933-43.

72. Wang B, et al., 1995 J. Virol, 21:102-12.

73. Boyer J D, et al., 1996, J. Med. Primatol., 25-242-50.

74. Boyer J D, et al., 1997, J. Infect. Dis., 176:1501-9.

75. Simon F, et al., Nature Medicine, 4:1032-1037.

76. Naldini, N., et al., 1996, Science, 272:263267; Srinivasakumar, N., et al., 1997, J. Tirol., 71:5841-5848; Zufferey, R., et al., 1997, Nature Biotechnology, 15:871-875; and Kim, V. N., et al, 1998, J. ViroL, 72:811-816.

77. Schwartz et al., 1992, J. Virol., 66:7176-7182; International Publication No. WO 93/20212 (1993); Schneider, R., et al., 1997, J. Virol., 71:4892-4903 (concerning the identification and mutation of inhibitory and instability regions using multiple point mutations within HIV-1 gag, protease and pol coding regions to reduce the effects of these regions and increase expression of the encoded polypeptide).

78. MacGregor et al., 1998, J. Infect Dis 178, 92-100.

79. Donnelly et al., 1997, Annu. Rev. Immunol. 15, 617-648.

80. Winzeler et al., 1998, Science 281, 1194-1197.

81. Ulmer et al., 1993, Science, 259, 1745-1749.

82. Georges-Courbot et al., 1998, J. Virol., 72, 600-608.

83. Santiago et al., 2001, Science, 295, 456-460.

84. Dalgleish et al. 1984, Nature, 312, 763-766.

85. Maddon et al., 1986, Cell, 47, 333-348.

86. Albert, et al. 1987, AIDS Res.

87. Desrosiers et al, 1989, AIDS Research and Human Retroviruses, 5:465-473.

88. Tsujimoto et al, Nature, 341, 539-541.

89. Fukasawa et al., 1989, Nature, 333, 457-541.

90. Courgnaud et al., 2001, J Virol, 75, 857-66.

91. Corbet et al, 2000, J. Virol. 74, 529.

92. Gao et al., 1999, Nature 397, 436-41.

93. Reitter, et al, 1998, Nat. Med., 4, 679-84.

94. Santiago, et al., 2003, 77, 2233-2242.

95. Syu, et al., 1991, J. Virol., 65, 6349-6352.

96. Souquiere, S., et al., 2001, J. Virol., 75, 7086-7096.

97. Price, A. M., et al., 2002, AIDS Res. Hum. Retrovir., 18, 657-660.

98. Sharp, P. M., et al., 2001, Phil. Trans. R. Soc. London. B Biol. Sci., 356, 867-876.

99. Ling, B., et al., 2003, J. Virol., 77, 2214-2226.

100. Thompson, J. D., et al., 1994, Nucleic Acids Res., 22, 4673-4680.

101. Vanden Haesevelde, M. M., et al., 1996, J. Virol., 221, 346-350.

102. Butynski, T. M., 2001, In Beck et al. (ed), Great Apes and Humans: the Ethics of Coexistence, Smithsonian Institute Press, Washington, D.C.

103. Selig et al., 1997, J. Virol., 71, 4824-4846.

104. Di Marzio, P. et al., 1995, J. Virol., 69, 7909-7916.

105. Karlin and Altschul, 1990, Proc. Natl. Acad. Sci. USA 87:2264-2268, modified as in Karlin and Altschul, 1993, Proc. Natl. Acad. Sci. USA 90:5873-5877.

106. Altschul et al, 1990, J. Mol. Biol. 215:403-410.

107. Altschul et al., 1997, Nucleic Acids Res. 25:3389-3402.

TABLE 1
Glycosylation in the SIVcpz versus HIV-1 Group M gp120 proteins

| | C1 | | | C2 | | C3 | V4 | C4 | V5 | C5 | |
|---|---|---|---|---|---|---|---|---|---|---|---|
| | region | V1 loop | V2 loop | region | V3 loop | region | region | region | region | region | TOTAL |
| TAN1 | 1 | 4 | 2 | 7 | 1 | 3 | 4 | 0 | 2 | 1 | 25 |
| ANT | 3 | 0 | 2 | 6 | 1 | 3 | 1 | 1 | 1 | 0 | 18 |
| US | 1 | 3 | 2 | 5 | 1 | 2 | 3 | 0 | 3 | 0 | 20 |
| GAB1 | 1 | 3 | 2 | 5 | 1 | 4 | 2 | 1 | 3 | 0 | 22 |
| GAB2 | 2 | 4 | 2 | 5 | 1 | 2 | 2 | 0 | 3 | 0 | 21 |
| CAM3 | 2 | 3 | 2 | 5 | 1 | 4 | 4 | 0 | 3 | 0 | 24 |
| CAM5 | 1 | 3 | 3 | 4 | 1 | 2 | 5 | 1 | 2 | 0 | 22 |
| mean | 1.6 | 2.9 | 2.1 | 5.3 | 1.0 | 2.9 | 3.0 | 0.4 | 2.4 | 0.1 | 21.71 |
| A-U455 | 1 | 5 | 3 | 5 | 0 | 3 | 3 | 0 | 3 | 0 | 23 |
| A-Q231 | 1 | 3 | 1 | 6 | 1 | 4 | 5 | 0 | 2 | 0 | 23 |
| B-JRFL | 1 | 4 | 3 | 4 | 1 | 4 | 4 | 0 | 2 | 0 | 23 |
| C-TH22 | 1 | 3 | 2 | 5 | 1 | 3 | 4 | 0 | 1 | 0 | 20 |
| C-UG26 | 2 | 3 | 1 | 7 | 1 | 4 | 4 | 0 | 2 | 0 | 24 |
| D-ELI | 2 | 3 | 3 | 6 | 0 | 2 | 7 | 0 | 4 | 0 | 27 |
| D-NDK | 2 | 1 | 2 | 6 | 0 | 1 | 5 | 0 | 3 | 0 | 20 |
| E-CM24 | 2 | 5 | 1 | 6 | 1 | 2 | 4 | 0 | 3 | 0 | 24 |
| E-TH02 | 2 | 4 | 2 | 7 | 0 | 2 | 4 | 1 | 3 | 0 | 25 |
| F1-BR0 | 1 | 4 | 2 | 6 | 1 | 3 | 3 | 1 | 3 | 0 | 24 |
| F2-MP2 | 2 | 3 | 2 | 5 | 1 | 5 | 4 | 0 | 1 | 0 | 23 |
| K-MP53 | 2 | 3 | 3 | 7 | 1 | 3 | 3 | 1 | 3 | 0 | 26 |
| G-SE61 | 2 | 6 | 2 | 7 | 1 | 4 | 4 | 0 | 3 | 0 | 29 |
| G-DRCB | 2 | 4 | 3 | 7 | 1 | 4 | 4 | 1 | 3 | 0 | 29 |
| H-VI99 | 1 | 5 | 3 | 6 | 1 | 3 | 4 | 0 | 2 | 0 | 25 |
| H-CF05 | 2 | 4 | 3 | 7 | 1 | 3 | 5 | 0 | 3 | 0 | 28 |
| J-SE78 | 2 | 4 | 1 | 6 | 1 | 3 | 3 | 1 | 3 | 0 | 24 |
| J-SE700 | 2 | 4 | 2 | 7 | 1 | 3 | 4 | 1 | 3 | 0 | 27 |
| mean | 1.7 | 3.8 | 2.2 | 6.1 | 0.8 | 3.1 | 4.1 | 0.3 | 2.6 | 0.0 | 24.67 |
| p-value | | 0.0704 | | 0.0375 | | | 0.0454 | | | | 0.0092 |

What is claimed:

1. An isolated nucleic acid comprising the nucleotide sequence of SEQ ID NO: 1, or a degenerate variant of SEQ ID NO: 1

2. The isolated nucleic acid of claim 1 where said nucleotide sequence is a derivative of SEQ ID NO: 1.

3. The isolated nucleic acid of claim 1 where said nucleotide sequence is complementary to SEQ ID NO: 1, or complementary to a fragment of SEQ ID NO: 1.

4. The isolated nucleic acid of claim 1 where said nucleotide sequence is complementary to a derivative of SEQ ID NO: 1.

5. The isolated nucleic acid sequence of claim 1 where said nucleotide sequence is at least 70% identical to the nucleotide sequence of SEQ ID NO: 1, or at least 70% identical to a degenerate variant of SEQ ID NO: 1.

6. An isolated nucleic acid sequence comprising a sequence that hybridizes under highly stringent conditions to a hybridization probe the nucleotide sequence of which consists of SEQ ID NO: 1, or a degenerate variant of SEQ ID NO: 1.

7. The isolated nucleic acid sequence of claim 6 where the hybridization probe has a nucleotide sequence which consists of a derivative of SEQ ID NO: 1.

8. An isolated nucleic acid comprising a sequence that encodes a polypeptide, the amino acid sequence of said polypeptide is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

9. The isolated nucleic acid of claim 8 where the amino acid sequence of said polypeptide is selected from the group consisting of SEQ ID NO: 2 with conservative amino acid substitutions, SEQ ID NO: 3 with conservative amino acid substitutions, SEQ ID NO: 4 with conservative amino acid substitutions, SEQ ID NO: 5 with conservative amino acid substitutions, SEQ ID NO: 6 with conservative amino acid substitutions, SEQ ID NO: 7 with conservative amino acid substitutions, SEQ ID NO: 8 with conservative amino acid substitutions, SEQ ID NO: 9 with conservative amino acid substitutions and SEQ ID NO: 10 with conservative amino acid substitutions.

10. A purified polypeptide comprising an amino acid sequence, the amino acid sequence of which is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

11. The purified polypeptide of claim 10 where said amino acid sequence is at least 70% identical to the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

12. A purified immunogenic peptide comprising an amino acid sequence of at least 10 consecutive residues, the amino acid sequence of which is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

13. A purified polypeptide comprising an amino acid sequence, the amino acid sequence of which is selected from the group consisting of SEQ ID NO: 2 with conservative amino acid substitutions, SEQ ID NO: 3 with conservative amino acid substitutions, SEQ ID NO: 4 with conservative amino acid substitutions, SEQ ID NO: 5 with conservative amino acid substitutions, SEQ ID NO: 6 with conservative amino acid substitutions, SEQ ID NO: 7 with conservative amino acid substitutions, SEQ ID NO: 8 with conservative amino acid substitutions, SEQ ID NO: 9 with conservative amino acid substitutions and SEQ ID NO: 10 with conservative amino acid substitutions

14. A purified polypeptide comprising an amino acid sequence, the amino acid sequence of which is selected from the group consisting of SEQ ID NO: 19, SEQ ID NO: 20 and SEQ ID NO: 21.

15. A composition comprising at least one polypeptide according to claim 10 in combination with a pharmaceutically acceptable carrier.

16. A composition comprising at least one polypeptide according to claim 12 in combination with a pharmaceutically acceptable carrier.

17. An antibody capable of binding to the polypeptide of claim 10.

18. An antibody capable of binding to the polypeptide of claim 14.

19. A kit for the detecting the presence of a virus of the SIVcpz type in a sample, said kit comprising an antibody according to claim 17 and reagents for the detection of the immunological complex formed between said antibody and said virus.

20. The kit of claim 19 where the virus is selected from the group consisting of SIVcpzTAN1, SIVcpzTAN2 and SIVcpzANT.

21. The kit of claim 19 where said kit comprises an antibody according to claim 18.

22. The kit of claim 21 where the virus is selected from the group consisting of SIVcpzTAN1, SIVcpzTAN2 and SIVcpzANT.

23. A method of detecting the presence of a virus of the SIVcpz type in a biological sample containing an antigen said virus comprising contacting the sample with the antibody of claim 14 under conditions that allow the formation of an antibody-antigen complex and detecting said complex.

24. The method of claim 23 where the virus is selected from the group consisting of SIVcpzTAN1, SIVcpzTAN2 and SIVcpzANT.

25. The method of claim 23 where the antibody is the antibody of claim 15.

26. The method of claim 25 where the virus is selected from the group consisting of SIVcpzTAN1, SIVcpzTAN2 and SIVcpzANT.

27. A method of detecting the presence of a virus of the SIVcpz type in a biological sample comprising contacting said sample with the nucleic acid of claim 1 and detecting said nucleic acid bound to the genomic DNA, mRNA or cDNA of the SIVcpz virus.

28. The method of claim 27 where the virus is selected from the group consisting of SIVcpzTAN1, SIVcpzTAN2 and SIVcpzANT.

29. A method of detecting the presence of a virus of the SIVcpz type in a biological sample comprising contacting said sample with the nucleic acid of claim 2 and detecting said nucleic acid bound to the genomic DNA, mRNA or cDNA of the SIVcpz virus.

30. The method of claim 29 where the virus is selected from the group consisting of SIVcpzTAN1, SIVcpzTAN2 and SIVcpzANT.

31. A vector comprising the nucleic acid of claim 1.

32. A vector comprising the nucleic acid of claim 2.

33. A cell comprising the vector of claim 31.

34. A cell comprising the vector of claim 32.


SEQUENCE LISTING (21 sequences)

SEQ ID NO: 1 - DNA, 9326 bp, Simian immunodeficiency virus
gctcttgcctaatctgccagatctgagcctgggagctctctggtagtggctggctagagaccgctgcttaacgctcaataaagcctgcctgagagtgttaacagtgtgtgcccatttcataccgcgtctgccctggggtagagatccctcagatttgtagtggctaagtaaaaatctctaccagtggcgcccgaacagggacttgagaagcagggaacgcggcccctggacgcaggactcggcttgtgacagcgcaatcacaagaggcgaggcggactccggtggtgagtacaaattttgttgtcggtgggcaaccctagaggaagggcgaagtctctaggtaacaggggaaatgggtgcgagagcgtcagtgttgaggggagataagctggatacatgggaatccataaggcttaaatccagaggcaggaaaaaatatttaataaaacatctagtatgggccggaagcgaactacagcgtttcgcgatgaatcccggtctcatggagaacgtagaaggctgctggaaaatcatcctccagctgcagccttcggtagacattggttctccagaaatcatttctttgtttaataccatctgtgtactctactgcgtacacgcaggagaaagagtccaagatacggaagaagcagtcaaaattgtgaaaatgaaactaactgtacagaaaaataactccacagcgacatctagtggacaaagacagaatgcaggtgaaaaagaggaaacagtgccacctagtggcaatacaggaaacacagggagagcaacagagacacctagtgggagtagactatacccagtgataactgatgcacagggagttgcaaggcatcagcctatttcacctagaactctaaatgcctgggtaagggtaatagaagaaaaagggtttaatccagaagtaataccaatgttctcagcattgtctgagggagcaaccccttatgatctaaatagtatgctcaatgctgttggggaacatcaagcagcaatgcaaatgttgaaggaagtcatcaatgaggaagcagcagagtgggacagagcacatcccgctcatgcaggaccccagcaagcagggatgctaagagagcccacaggggcagatattgcagggaccactagtacgctacaagaacaagtactgtggatgacaaccccacaggcacaaggaggagtgccagtaggagacatctataaaaggtggataattttaggattaaataaattagtcagaatgtacagccctgttagcattttggacataaaacagggaccaaaagaaccattcagagattatgtagacagattctacaaaacaatcagagcagaacaagcatctcaaccagtaaaaacttggatgacagaaactttactggtacaaaatgcaaacccagattgtaagcatatcttaaaagccttggggcaaggagcaacattagaagaaatgctcacagcctgtcaaggagtgggaggaccctctcataaggcaaagattctggctgaagcaatggcctcagcaacagcagggggagtaaatatgctgcagggaggaaaaagaccacccttaaaaaagggtcagctgcagtgttttaactgtgggaaagtaggccatacagcaagaaattgtagggctccaagaaagaaaggttgctggaggtgtggacaagagggacatcaaatgaaggactgcaccaccagaaacaacagcactggggtaaattttttagggaaacgcacccccttgtgggggtgcagaccagggaactttgtgcagaacaccccagagaaagggaaggctcaggagcaggagacagcacagacaccagtggtgccaactgccccaccactggagatgacgatgaaaggcgggttctccctcaagtcaatctttggcagcgaccaatgatgacagtaaaagtccagggacaagtctgtcaagctcttttagatactggagcagatgacagtgttttttgtaacatcaaattaaagggacagtggacaccaaaaaccataggaggaataggaggatttgtaccagttagtgagtactataatattccagtacaaattggcaataaagaagtcagagccactgtcctagtgggagaaacccccattaatataataggtagaaatattttaaagcaattaggatgtaccttaaattttcctattagcccaatagaggtagtaaaagtacaattaaaagaaggaatggatgggccaaaagtaaagcagtggcccctctccaaggagaaaattgaggcattaacagaaatatgtaagacattggaaaaggaaggaaaaatttctgcagttggaccagaaaacccatataacacaccaatttttgccattaagaaaaaggatacctctaaatggagaaaattagtagatttcagagaactgaataaaagaactcaagatttttgggagttacagctaggaataccccatccggcagggttaagaaaaagaaatatggtgacagtactggatgtaggggatgcctacttttccattcccctggatccagacttcagaaagtatacagcttttaccatacccagtctcaataataacacaccagggaaaagatttcagtataacgtgttacctcaaggttggaagggatctccagcaatttttcagagcagtatgacaaaaatcctagatcctttcagaaaagaacacccagatgtggacatttaccaatatatggatgatctttacataggttcagatcttaatgaagaggaacataggaaactgataaagaagctgagacagcatctgttaacatggggattagagacccctgacaaaaagtatcaggaaaaacctccattcatgtggatgggctatgagctacatccaaataaatggacagttcaaaatatcacattaccagaaccagagcagtggacagtgaatcatatccagaagttggtaggcaaacttaattgggccagtcaaatttatcatggaataaaaactaaagaactatgcaaattgattagaggagtaaaaggattaactgagccagtagaaatgaccagggaagcagaattggagttagaagaaaataagcagattctaaaagaaaaggttcaaggagcatactatgatcctaaattacctctgcaagcagcaatacagaagcaggggcaaggacagtggacatatcagatatatcaggaagaagggaaaaatttaaaaacaggaaaatatgcaaaatcaccaggtacccacaccaatgagataagacaattagcaggactgatacagaaaataggcaatgagagcataataatttggggtattgtgcctaaatttttattacctgtatccaaagagacatggagccagtggtggactgattactggcaagttacctgggtacctgagtgggaatttattaacaccccaccactaatcaggctatggtacaatctgttgtctgaccccatcccagaagcagaaaccttttatgtagatggggcagcaaacagagacagtaaaaagggaagagcaggatatgtaacaaacagaggcagatacaggtcaaaggacttagagaacaccactaatcaacaagcagaattatgggcagtagatctagccttaaaagactcaggagcacaggtaaatatagtcacagattcccaatatgttatgggagttttacagggattaccagatcaaagtgactcccccatagtagagcaaattattcaaaagttaacacaaaagacagcaatttatctagcatgggtaccagcccataaaggtatagggggtaatgaagaagtagacaaattggttagtaaaaatattagaaaaatattattcctggatggaattaatgaagcacaggaagaccatgataaatatcacagtaattggaaagctttagctgatgaatataatctgcccccagttgtggctaaagaaattattgctcagtgtccaaaatgccatataaaaggagaggctatacatggacaggtggactacagtccagaaatctggcaaatagactgtacccacctagaaggaaaggtcatcatagtagcagtgcatgtagctagtggtttcatagaagcagaagtcataccagaagaaacaggaagagaaaccgcttacttcatcctaaaattggcaggaagatggcctgtaaagaaaatacatacagataatggaccaaattttactagtacagcagtgaaggcagcctgctggtgggcacaaattcaacatgaatttgggattccatataatcctcaaagtcaaggagtagtagaatctatgaataaacaattaaagcaaattatagagcaagtcagggaccaagcagagcaactgaggacagcagtaatcatggcagtgtatatccacaattttaaaagaaaaggggggattggggagtacactgcaggggaaagactattagacatactaactacaaatatacagacaaaacaattacaaaaacaaattttaaaagttcaaaattttcgggtttattatagggacgccagagatccaatttggaagggaccagcgcgactactgtggaaaggtgaaggggcagtagtaataaaagaaggagaagacattaaagtagtacccaggagaaaagcaaaaatcataaaagagtatggaaaacagatggcaggtgcaggtggtatggatgatagacagaatgagacttagaacatggacaagcctagttaaacatcatatctttacaaccaaatgctgtaaagattggaagtatagacatcattatgaaactgatacaccaaaaagagcaggggaaatacacatacctctaacagaaagatcaaaattagtggttttacattattggggtctagcctgtggagaaagaccatggcatctaggtcatggcataggattagaatggagacaaggaaaatacagtacacaaatagaccctgaaacagcagaccaattgattcacactaggtattttacctgttttgctgcaggagcagttcggcaagcaatattaggagaaagaatattgacattctgccactttcaatcaggacacagacaggtagggactctgcaattcttagctttcagaaaggtagttgagagccaagataaacagccaaagggaccaaggaggcccttgccatctgttacaaaactaacagaggacagatggaacaagcaccgaacgacaacgggccgcagagagaaccatacactgagtggctgttagacatcctagaagaaataaaacaagaagcagtgaaacactttccaagaccaatattacagggggtaggaaattgggtcttcaccatttatggagactcctgggagggagtacaggaattaatcaagatcttgcagagagctttgtttacccactatcgccatggttgtatccacagcagaataggatcatgaatcccatagatcctcaggtagcaccatgggaacatccaggagctgcacctgaaacaccttgtacaaactgttactgtaaaaaatgctgctttcattgcccagtttgctttacgaaaaaagcattaggaatctcctatggcaggaagagaagaggacgcaaatctgctgtacacagtacgaataatcaagatcctgtacgacagcagtaagtacccatgataaaaatagtagtgggaagtgtgtcaactaatgtcataggcattctttgtatattactgattttaatagggggaggcttgctaataggtataggtataagaagagagttagaaagggaaaggcaacatcaaagagtattagaaaggctagctagaagattaagcatagacagtggagtagaagaagatgaagaatttaattggaataactttgatcctcataattacaatcctagggattggatttagcacttattacaccacagtgttttatggagtacctgtttggaaagaggcccaaccaaccttgttttgtgcctctgatgctgatattactagtagagataaacacaacatatgggcaacacataactgtgtgcctttagatcccaatccttatgaagtaaccctagccaatgtgtcaataaggtttaatatggaagaaaattacatggtgcaagagatgaaagaagatatattatcactttttcaacagagttttaagccttgtgtaaaattaacaccattttgcataaagatgacatgtacaatgactaataccacaaataaaaccctgaattcggcaacaacaaccttaacaccaacagtaaatttgagttctatacctaactatgaggtgtataattgttcatttaatcagacaactgagtttagagataagaaaaaacaaatatattccttgttttatagagaagatattgtaaaagaggatggtaacaataatagttattatttacataattgcaatacctcagtcattactcaagaatgtgataaatctacttttgaaccaattcccatcagatactgtgctccagcaggctttgccctgttaaaatgtagagatcagaatttcacagggaaaggacaatgctccaatgtctcagtagttcactgtacacatgggatttatcctatgatagccacagcattacacttaaatgggtccctggaagaagaagaaacaaaagcttactttgttaatacctcagttaatacacccttattagtaaaatttaatgtatcaataaatttaacgtgtgaaagaacaggaaacaatacaagaggtcaagtacagataggtccaggtatgaccttttataatatagaaaatgtagtaggggacaccaggaaagcttattgttcagtcaatgcaacaacatggtacaggaacttagattgggctatggctgccataaacacaaccatgagggccagaaatgaaacggtacaacaaacgttccaatggcagagggatggagaccctgaggtcactagcttctggttcaattgtcaaggagaattcttttactgtaatctcacaaattggactaatacctggacagctaatagaaccaataatactcatggtactcttgttgcaccatgcagactgaggcagatagtaaatcattggggtatagtgtcaaaaggggtttaccttcccccaaggaggggaacagtaaaatgtcactcaaacatcacaggacttatcatgacagcagaaaaagacaacaataatagttataccccccaattttctgctgtagtagaagactattggaaagtagaattagcaagatataaagtggtggaaattcagcccttgtcagtggctccaaggccaggaaaaaggcctgaaattaaggccaatcatactaggtcaagaagagatgtgggcataggactgttgtttcttggatttcttagtgcagcaggaagtacaatgggcgcagcgtcaatagcgctgacggcacaggccagaggattactctctggtattgtacagcagcaacaaaacctgcttcaggccatagaagcgcaacaacacttgttgcagctctctgtatggggcattaagcagctccaggccagaatgcttgcagtagagaaatacataagagaccaacagctcctaagcctctggggatgtgctaacaaattggtgtgtcacagtagtgtgccatggaacctcacctgggctgaagattctacaaagtgcaatcacagtgatgcaaagtactatgactgtatatggaacaatttgacttggcaggaatgggatcgattagtagaaaactctacaggaaccatatactccctgttagagaaagcacaaacacaacaggagaaaaacaaacaagagttgttagaattagacaaatggagcagtctttgggattggtttgatataacacaatggctgtggtatataaaaatagctataatcatagtagcaggattagtaggacttagaattctcatgtttatagttaatgtagttaagcaagttaggcagggttatacacccctattttcacagatccctacccaagcggagcaggatccagaacagccaggaggaatcgcaggaggaggtggaggcagagacaacatcaggtggacgccctcgccagcaggattcttcagtatcgtctgggaggacctcaggaacctcctcatctggatataccagacctttcaaaacttcatctggatcctctggatcagcctgcaagcactgaaacaggggataatcagcttggcacacagcctagtaatagtgcatagaactatcatagtaggagttagacagatcattgagtggagcagtaatacttatgctagcttaagagttttgctaatacaagccatagacagacttgctaactttacagggtggtggacagatttaatcatagaaggagtggtttacatagccaggggaatcagaaatattcctagaagaattagacagggtctggaactagccttaaattaaaatgggaaacatatttggtagatggcctggggcccggaaagccatcgaagatcttcataacacctcaagtgagcctgtaggacaggcctcacaagacctccagaataaaggaggtctcactactaacaccctaggtacctcagcagatgtgttagaatactctgcagaccatactgaagaagaagtaggttttccagtcagaccagcagtacccatgagacccatgacagagaagctagcaatagatctgtcatggttcttaaaagaaaaggggggactggatgggctatttttctctccaaaaagagcagccatcctagacacctggatgtataatacacagggtgtctttccagactggcagaactacacccctggaccaggaatcagatacccactgtgtaggggatggttatttaagttggtaccggtagacccaccagaagatgatgagaagaacatcttgctacatccagcctgtagccatggaactaccgatccagatggagagactctgatctggcgctttgacagcagcctagcaagaaggcacatagccagagaaagatatccggagtacttcaaataaggacttccgggtgccatgactcagaactgctgacagaggacttttggactcgggactttccaatgtgggtggttactgggcgggacaggggagtggttttgcccgctgagctgcatataagcagctgctttgcgctctgtaaaggctcttgcctaatctgccagatctgagcctgggagctctctggtagtggctggctagagaccgctgcttaacgctcaataaagcctgcctgagagtgtta

SEQ ID NO: 2 - protein, 524 aa, Simian immunodeficiency virus
MGARASVLRGDKLDTWESIRLKSRGRKKYLIKHLVWAGSELQRFAMNPGLMENVEGCWKIILQLQPSVDIGSPEIISLFNTICVLYCVHAGERVQDTEEAVKIVKMKLTVQKNNSTATSSGQRQNAGEKEETVPPSGNTGNTGRATETPSGSRLYPVITDAQGVARHQPISPRTLNAWVRVIEEKGFNPEVIPMFSALSEGATPYDLNSMLNAVGEHQAAMQMLKEVINEEAAEWDRAHPAHAGPQQAGMLREPTGADIAGTTSTLQEQVLWMTTPQAQGGVPVGDIYKRWIILGLNKLVRMYSPVSILDIKQGPKEPFRDYVDRFYKTIRAEQASQPVKTWMTETLLVQNANPDCKHILKALGQGATLEEMLTACQGVGGPSHKAKILAEAMASATAGGVNMLQGGKRPPLKKGQLQCFNCGKVGHTARNCRAPRKKGCWRCGQEGHQMKDCTTRNNSTGVNFLGKRTPLWGCRPGNFVQNTPEKGKAQEQETAQTPVVPTAPPLEMTMKGGFSLKSIFGSDQ

SEQ ID NO: 3 - protein, 999 aa, Simian immunodeficiency virus
FFRETHPLVGVQTRELCAEHPREREGSGAGDSTDTSGANCPTTGDDDERRVLPQVNLWQRPMMTVKVQGQVCQALLDTGADDSVFCNIKLKGQWTPKTIGGIGGFVPVSEYYNIPVQIGNKEVRATVLVGETPINIIGRNILKQLGCTLNFPISPIEVVKVQLKEGMDGPKVKQWPLSKEKIEALTEICKTLEKEGKISAVGPENPYNTPIFAIKKKDTSKWRKLVDFRELNKRTQDFWELQLGIPHPAGLRKRNMVTVLDVGDAYFSIPLDPDFRKYTAFTIPSLNNNTPGKRFQYNVLPQGWKGSPAIFQSSMTKILDPFRKEHPDVDIYQYMDDLYIGSDLNEEEHRKLIKKLRQHLLTWGLETPDKKYQEKPPFMWMGYELHPNKWTVQNITLPEPEQWTVNHIQKLVGKLNWASQIYHGIKTKELCKLIRGVKGLTEPVEMTREAELELEENKQILKEKVQGAYYDPKLPLQAAIQKQGQGQWTYQIYQEEGKNLKTGKYAKSPGTHTNEIRQLAGLIQKIGNESIIIWGIVPKFLLPVSKETWSQWWTDYWQVTWVPEWEFINTPPLIRLWYNLLSDPIPEAETFYVDGAANRDSKKGRAGYVTNRGRYRSKDLENTTNQQAELWAVDLALKDSGAQVNIVTDSQYVMGVLQGLPDQSDSPIVEQIIQKLTQKTAIYLAWVPAHKGIGGNEEVDKLVSKNIRKILFLDGINEAQEDHDKYHSNWKALADEYNLPPVVAKEIIAQCPKCHIKGEAIHGQVDYSPEIWQIDCTHLEGKVIIVAVHVASGFIEAEVIPEETGRETAYFILKLAGRWPVKKIHTDNGPNFTSTAVKAACWWAQIQHEFGIPYNPQSQGVVESMNKQLKQIIEQVRDQAEQLRTAVIMAVYIHNFKRKGGIGEYTAGERLLDILTTNIQTKQLQKQILKVQNFRVYYRDARDPIWKGPARLLWKGEGAVVIKEGEDIKVVPRRKAKIIKEYGKQMAGAGGMDDRQNET

SEQ ID NO: 4 - protein, 198 aa, Simian immunodeficiency virus
MENRWQVQVVWMIDRMRLRTWTSLVKHHIFTTKCCKDWKYRHHYETDTPKRAGEIHIPLTERSKLVVLHYWGLACGERPWHLGHGIGLEWRQGKYSTQIDPETADQLIHTRYFTCFAAGAVRQAILGERILTFCHFQSGHRQVGTLQFLAFRKVVESQDKQPKGPRRPLPSVTKLTEDRWNKHRTTTGRRENHTLSGC

SEQ ID NO: 5 - protein, 83 aa, Simian immunodeficiency virus
MEQAPNDNGPQREPYTEWLLDILEEIKQEAVKHFPRPILQGVGNWVFTIYGDSWEGVQELIKILQRALFTHYRHGCIHSRIGS

SEQ ID NO: 6 - protein, 136 aa, Simian immunodeficiency virus
MNPIDPQVAPWEHPGAAPETPCTNCYCKKCCFHCPVCFTKKALGISYGRKRRGRKSAVHSTNNQDPVRQQSLPKRSRIQNSQEESQEEVEAETTSGGRPRQQDSSVSSGRTSGTSSSGYTRPFKTSSGSSGSACKH

SEQ ID NO: 7 - protein, 105 aa, Simian immunodeficiency virus
MAGREEDANLLYTVRIIKILYDSNPYPSGAGSRTARRNRRRRWRQRQHQVDALASRILQYRLGGPQEPPHLDIPDLSKLHLDPLDQPASTETGDNQLGTQPSNSA

SEQ ID NO: 8 - protein, 83 aa, Simian immunodeficiency virus
MIKIVVGSVSTNVIGILCILLILIGGGLLIGIGIRRELERERQHQRVLERLARRLSIDSGVEEDEEFNWNNFDPHNYNPRDWI

SEQ ID NO: 9 - protein, 871 aa, Simian immunodeficiency virus
MKNLIGITLILIITILGIGFSTYYTTVFYGVPVWKEAQPTLFCASDADITSRDKHNIWATHNCVPLDPNPYEVTLANVSIRFNMEENYMVQEMKEDILSLFQQSFKPCVKLTPFCIKMTCTMTNTTNKTLNSATTTLTPTVNLSSIPNYEVYNCSFNQTTEFRDKKKQIYSLFYREDIVKEDGNNNSYYLHNCNTSVITQECDKSTFEPIPIRYCAPAGFALLKCRDQNFTGKGQCSNVSVVHCTHGIYPMIATALHLNGSLEEEETKAYFVNTSVNTPLLVKFNVSINLTCERTGNNTRGQVQIGPGMTFYNIENVVGDTRKAYCSVNATTWYRNLDWAMAAINTTMRARNETVQQTFQWQRDGDPEVTSFWFNCQGEFFYCNLTNWTNTWTANRTNNTHGTLVAPCRLRQIVNHWGIVSKGVYLPPRRGTVKCHSNITGLIMTAEKDNNNSYTPQFSAVVEDYWKVELARYKVVEIQPLSVAPRPGKRPEIKANHTRSRRDVGIGLLFLGFLSAAGSTMGAASIALTAQARGLLSGIVQQQQNLLQAIEAQQHLLQLSVWGIKQLQARMLAVEKYIRDQQLLSLWGCANKLVCHSSVPWNLTWAEDSTKCNHSDAKYYDCIWNNLTWQEWDRLVENSTGTIYSLLEKAQTQQEKNKQELLELDKWSSLWDWFDITQWLWYIKIAIIIVAGLVGLRILMFIVNVVKQVRQGYTPLFSQIPTQAEQDPEQPGGIAGGGGGRDNIRWTPSPAGFFSIVWEDLRNLLIWIYQTFQNFIWILWISLQALKQGIISLAHSLVIVHRTIIVGVRQIIEWSSNTYASLRVLLIQAIDRLANFTGWWTDLIIEGVVYIARGIRNIPRRIRQGLELALN

SEQ ID NO: 10 - protein, 195 aa, Simian immunodeficiency virus
MGNIFGRWPGARKAIEDLHNTSSEPVGQASQDLQNKGGLTTNTLGTSADVLEYSADHTEEEVGFPVRPAVPMRPMTEKLAIDLSWFLKEKGGLDGLFFSPKRAAILDTWMYNTQGVFPDWQNYTPGPGIRYPLCRGWLFKLVPVDPPEDDEKNILLHPACSHGTTDPDGETLIWRFDSSLARRHIARERYPEYFK

SEQ ID NO: 11 - DNA, 23 bp, Simian immunodeficiency virus
  misc_feature (6)..(6): n = a or g or c or t/u, unknown or other base
ccagcncacaaaggnataggagg

SEQ ID NO: 12 - DNA, 21 bp, Simian immunodeficiency virus
  misc_feature (9)..(9): n = a or g or c or t/u, unknown or other base
acbacygcnccttchcctttc

SEQ ID NO: 13 - DNA, 26 bp, Simian immunodeficiency virus
ggaagtggatacttagaagcagaagt

SEQ ID NO: 14 - DNA, 27 bp, Simian immunodeficiency virus
cccaatccccccttttcttttaaaatt

SEQ ID NO: 15 - DNA, 688 bp, Simian immunodeficiency virus
ccaagcgcagcaggatccagaacagcccggaggaatcgcagaaggaggtggaggcagaggcaacatcaggtggacgccctcgccaacaggattcttcagtatcgtctgggaggacctcaggaacctcctcatctggctctaccagacctgtcgaaacttcatctgggtcctgtggacgatcctgcaagcactgaaacaggggacaatcagcctagcaaacaacctagtaatagtgcatagatatatagtagtaaaaattagacaaattattgagtggtgtcacaatacttatgctagtttaagagcttcgctgatacatgcaatagacagacttgctgactttacagggtggtggacagacttaatcatagaaggaataacatacataggcaggggaatcagaaacatccctagaaggatcagacagggtctagaaatagccttaaattaaaatgggaaacatctttggtagatggcctggagctcgaagagctattgaagatcttcataaaagctcacatgagcctataggacaggcctcaacagacctccaaaatagagggggcttaaccaacaacaccataggtacttcagcagatgtagtagagtattctgcagaccatactgaggaagaagtagggtttccagttagaccagcagtacccatgagacccatgacagaaacacg

SEQ ID NO: 16 - protein, 227 aa, Simian immunodeficiency virus
QAQQDPEQPGGIAEGGGGRGNIRWTPSPTGFFSIVWEDLRNLLIWLYQTCRNFIWVLWTILQALKQGTISLANNLVIVHRYIVVKIRQIIEWCHNTYASLRASLIHAIDRLADFTGWWTDLIIEGITYIGRGIRNIPRRIRQGLEIALNMGNIFGRWPGARRAIEDLHKSSHEPIGQASTDLQNRGGLTNNTIGTSADVVEYSADHTEEEVGFPVRPAVPMRPRQKH

SEQ ID NO: 17 - DNA, 335 bp, Simian immunodeficiency virus
gtggatacttagaagcagaagtcataccagaagaaacaggaagggaaacagcttatttcatcttaaaattggctggaagatggcctgtaaagaaaatacatacagataatgggccaaactttactagtgcagcagtaaaagcagcctgttggtgggcacaaatccaacatgaatttgggattccatataatcctcaaagtcaaggagtagtagaatccatgaataaacaattaaagcaaattatagaacaaattagggaacaagcagagcacctgaggacagcagtggctatggcagtgtatatccacaattttaaaagaaaaggggggatgggg

SEQ ID NO: 18 - protein, 111 aa, Simian immunodeficiency virus
GYLEAEVIPEETGRETAYFILKLAGRWPVKKIHTDNGPNFTSAAVKAACWWAQIQHEFGIPYNPQSQGVVESMNKQLKQIIEQIREQAEHLRTAVAMAVYIHNFKRKGGMG

SEQ ID NO: 19 - protein, 5 aa, Simian immunodeficiency virus
KGPRR

SEQ ID NO: 20 - protein, 11 aa, Simian immunodeficiency virus
CNHSDAKYYDC

SEQ ID NO: 21 - protein, 10 aa, Simian immunodeficiency virus
CAKNSSDIQC